(19) United States
(12) Patent Application Publication
(10) Pub. No.: US 2014/0107523 A1
(43) Pub. Date: Apr. 17, 2014

(54) MODEL FOR TRAUMATIC BRAIN INJURY

(71) Applicant: University of Rochester, Rochester, NY (US)

(72) Inventors: Anthony L. Petraglia, West Henrietta, NY (US); Jason H. Huang, Pittsford, NY (US)

(73) Assignee: University of Rochester, Rochester, NY (US)

(21) Appl. No.: 14/051,410

(22) Filed: Oct. 10, 2013

Related U.S. Application Data
(60) Provisional application No. 61/713,376, filed on Oct. 12, 2012.

(51) Int. Cl. A61D 3/00; A61B 5/00; A61B 9/00

(52) U.S. Cl. CPC A61D 3/00; A61B 9/00; A61B 5/4064
USPC 600/553

(57) ABSTRACT

Models for simulating traumatic brain injury provide analogues for studying similar occurrences in humans. Such models may include inserting a subject into a sheath. While in the sheath, the subject may be placed on a pad beneath an impactor. The head of the subject may be impacted by the impactor, whereby the head of the subject moves into the pad. Methods may further include attaching a helmet to the head of the subject. An arm of an impact device may be connected to a handle of the helmet, such that the head of the subject is restrained relative to the impact device. While advancing the impactor, the arm may be disconnected from the handle, such that the head of the subject becomes unrestrained relative to the impact device and moves into the pad in response to the impacting.

### RELATED APPLICATIONS

This application claims the benefit of U.S. Provisional Application No. 61/713,376, filed Oct. 12, 2012, the entire contents of which are incorporated by reference, as if fully set forth herein.

### GOVERNMENT LICENSE RIGHTS

This invention was made with government support under Grant No. NIH-R25-NS-065748 awarded by the National Institutes of Health and Grant No. NIH-R01-NS-067435 awarded by the National Institutes of Health. The Government has certain rights to this invention.

### BACKGROUND

There has been increasing attention focused on the neurological sequelae of sports-related traumatic brain injury (“TBI”), particularly concussion and subconcussion. In the United States alone, an estimated 1.6 to 3.8 million sports-related concussions occur each year. Most studies have focused on moderate and severe TBI, but concussive and sub-concussive head injuries affect more people, occur more frequently, and are a silent epidemic of increasing importance. Additionally, there is a greater appreciation that repetitive mild TBI may lead to detrimental effects on neurological function both short- and long-term, including the potential for chronic neurodegenerative syndromes such as chronic traumatic encephalopathy (“CTE”).

The syndrome of CTE is now appreciated to occur not only in football, but also in boxers, wrestlers, hockey players and even military personnel. The phenomenon is caused by episodic and repetitive blunt force impacts to the head and transfer of acceleration-deceleration forces to the brain. The disease presents clinically as a composite syndrome of mood disorders, neuropsychiatric disturbance and cognitive impairment, with or without sensori-motor impairment. Behavioral abnormalities such as judgment issues, increased risk-taking, and depression are characteristic and prominent early in the disease course. Additional symptoms may include difficulty sleeping, poor concentration and memory impairment. Definitive and confirmatory diagnoses of CTE still remain direct tissue histochemical and immunohistochemical analyses.

Brain damage due to traumatic impacts includes concussion, contusion, ischemia, edema, and enhanced intracranial pressure to exacerbate brain tissue/neuronal damage. Such injuries may be studied in an experimental setting, in which an animal model is used as an analog for traumatic injuries to a human brain.

TBI includes four overlapping phases, which include primary injury, evolution of the primary injury, secondary or additional injury, and regeneration. Primary injury to the brain can be induced by numerous mechanisms, which include the following major categories: 1) direct contusion of the brain from the skull; 2) brain contusion caused by a movement against rough interior surfaces of the skull, and/or indirect (contracoup) contusion of the brain opposite the side of the impact; 3) shearing and stretching of the brain tissue caused by motion of the brain structures relative to the skull and each other; 4) vascular response to the impact including subdural hematoma produced by rupture of bridging blood vessels located between brain and dura mater; decreased blood flow due to increased intracranial pressure or infarction; and brain edema caused by increased permeability of cerebral blood vessels. Diffuse axonal injury has been recognized as one of the main consequences of blunt head trauma; it is characterized by morphological and functional damages of axons throughout the brain and brainstem and leads to diffuse degeneration of cerebral white matter. Secondary injury mechanisms include complex biochemical and physiological processes, which are initiated by the primary insult and manifest over a period of hours to days. It has been established that such secondary injury may significantly contribute to post-traumatic neurological disability.

Experimental models of traumatic brain injury replicate certain pathological components or phases of clinical trauma in experimental animals aiming to address pathology and/or treatment. Models may address biomechanical aspects of brain injury or may be targeted toward improving the understanding of complex molecular detrimental cascades initiated by trauma. For example, experimental models for brain damage may be prepared for study of neuronal and/or vascular impairments, evaluation of drug efficacy against brain impairments/damage, memory-related learning, direct analysis (e.g., by magnetic resonance imaging), etc. For example, after applying a traumatic impact, brain damage of a subject may be evaluated on a time-related basis with MRI and/or MRI-based cerebrovascular flowmetry whereupon these parameters serve as analytical indexes of drug efficacy for brain damage, memory-associated learning, and/or behavioral responses. Other tests and analyses may be performed, such as memory-relates maze trials.

### SUMMARY

Prior laboratory studies regarding the consequences of repetitive head injury have provided data; however, none have adequately modeled and encapsulated the syndrome of CTE. Additionally, most experimental TBI utilizes anesthesia during the delivery of repetitive injury, which confounds the pathophysiology and post-traumatic outcome through both neuroprotective and neurodestructive pathways. Ascertainment bias is a major limitation in establishing causation, and not just association, between mild repetitive TBI and CTE. Thus, an animal model of CTE is essential for learning about the pathophysiological link between repetitive head injury and the development of chronic neurodegenerative disease; as well as important for discovering biomarkers of disease, improved diagnostic tools, and translational treatment approaches.

The subject technology includes a model of closed-head injury that utilizes some of the best facets of the most widely used animal models of TBI. In addition, to make the model as clinically relevant as possible, the number of hits was increased, the inter-injury interval was decreased, and the injuries were performed all in un-anesthetized mice. The combination of these variables in the model of the subject technology results in the neurobehavioral spectrum of disease observed in CTE, including cognitive issues, increased risk-taking, depression and sleep disturbances, as well as the histopathological hallmarks of CTE, including increased astrogliosis, microglial activation, and hyperphosphorylated tau protein accumulation.

TBI models according to embodiments of the subject technology allow for controlled closed-head impacts to un-anesthetized animals (e.g., mice). In one example, 280 12-week old mice were divided into control, single mild-TBI, and repetitive mild-TBI groups. Repetitive mild-TBI mice received six concussive impacts daily, for seven days. Behavior was assessed at various time-points. Neurological severity score (“NSS”) was computed and vestibulo-motor function tested with the wire grip test (“WGT”). Cognitive function was assessed with Morris water maze (“MWM”; anxiety/risk-taking behavior with the elevated-plus-maze (“EPM”); and depression with the forced swim/tail suspension tests. Sleep EEG/EMG studies were performed at one month.

It was found that NSS was elevated compared to controls in both TBI groups and improved over time. Repetitive mild-TBI mice demonstrated transient vestibulo-motor deficits on WGT. Repetitive mild-TBI mice demonstrated deficits in MWM acutely and persisting out to 6-months. Both mild-TBI groups demonstrated increased anxiety at 2-weeks but repetitive mild-TBI mice develop increased risk-taking behaviors at 1-month that persists at 6-months. Repetitive mild-TBI mice exhibit depression at 1-month. Both mild-TBI groups demonstrate sleep disturbances.

Described herein are the neurological sequelae of repetitive mild-TBI in models of the subject technology. The behavior exhibited by repetitive mild-TBI mice, in a model, resembles the characteristic neuro-psychiatric behavioral picture seen in CTE.

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into any independent clause, e.g., clause 1 or clause 55. The other clauses can be presented in a similar manner.

1. A method of modeling traumatic brain injury, comprising:
inserting a subject into a sheath, such that a head of the subject is restrained relative to a body of the subject;
placing the subject, while in the sheath, on a pad beneath an impactor;
impacting the head by the impactor, such that the head moves into the pad in response to the impacting.

2. The method of clause 1, wherein the subject is not anesthetized.

3. The method of clause 1, wherein the head is unbound relative to the pad during the impacting.

4. The method of clause 1, wherein the inserting comprises:
placing the subject through a first opening at the first end of the sheath; and
closing the first end to limit retraction of the subject through the first opening.

5. The method of clause 1, further comprising, prior to the impacting, placing a helmet on the head, the helmet comprising an impact plate.

6. The method of clause 5, further comprising, prior to the impacting, aligning the impact plate at an impact location at the head.

7. The method of clause 6, wherein the head is impacted by transferring a force received from the impactor through the impact plate to the head.

8. An impact model system, comprising:
a pad configured to support a subject and allow a range of travel by a head of the subject into the pad after an impact;
an impactor having a tip configured to travel from an initial location to an impact location at the head;
a containment sheath configured to restrain the head relative to a body of the subject.

9. The impact model system of clause 8, wherein the sheath comprises a first opening at a first end of the sheath, configured to receive the subject into the sheath.

10. The impact model system of clause 9, wherein the first opening is configured to be closed to prevent retraction through the first opening.

11. The impact model system of clause 8, wherein the sheath comprises a second opening at a second end of the sheath, configured to provide an airway for the subject.

12. The impact model system of clause 8, further comprising a helmet configured to be placed on the head and received the impact from the impactor.

13. The impact model system of clause 12, wherein the helmet comprises an impact plate configured to be aligned at the impact location.

14. The impact model system of clause 12, wherein the helmet comprises a band configured to secure the helmet to the head.

15. The impact model system of clause 12, wherein the helmet comprises a handle configured to receive a magnetic connection.

16. The impact model system of clause 15, further comprising an arm configured to form a magnetic connection with the handle.

17. The impact model system of clause 16, further comprising an arm configured to release the magnetic connection upon advancement of the impactor from the initial location to the impact location.

18. A method, comprising:
placing a subject on a pad beneath an impactor, the subject having a helmet attached to a head of the subject;
connecting an arm of an impact device to a handle of the helmet, such that the head is restrained relative to the impact device;
impacting the head by advancing the impactor from an initial location to an impact location;
while advancing the impactor, disconnecting the arm from the handle, such that the head becomes unrestrained relative to the impact device and moves into the pad in response to the impacting.

19. The method of clause 18, wherein the subject is not anesthetized.

20. The method of clause 18, wherein the connecting comprises applying a magnetic force between the arm and a handle.

21. The method of clause 20, wherein the disconnecting comprises applying a force to the arm that exceeds the magnetic force.

Additional features and advantages of the subject technology will be set forth in the description below, and in part will be apparent from the description, or may be learned by practice of the subject technology. The advantages of the subject technology will be realized and attained by the structure particularly pointed out in the written description and claims hereof as well as the appended drawings.

It is to be understood that both the foregoing general description and the following detailed description are exemplary and explanatory and are intended to provide further explanation of the subject technology as claimed.

### BRIEF DESCRIPTION OF THE DRAWINGS

The accompanying drawings, which are included to provide further understanding of the subject technology and are incorporated in and constitute a part of this specification, illustrate aspects of the subject technology and together with the description serve to explain the principles of the subject technology.

FIG. 1 shows an exemplary system for inducing traumatic brain injury in an animal, according to some embodiments of the subject technology.

FIGS. 2A, 2B, and 2C show stages of an exemplary system for restraining an animal, according to some embodiments of the subject technology.

FIG. 3A shows an exemplary helmet for use on an animal, according to some embodiments of the subject technology.

FIG. 3B shows an exemplary helmet for use on an animal, according to some embodiments of the subject technology.

FIG. 4A shows an exemplary system for inducing traumatic brain injury with an animal in a restrained state, according to some embodiments of the subject technology.

FIG. 4B shows an exemplary system for inducing traumatic brain injury with an animal in an unrestrained state, according to some embodiments of the subject technology.

FIG. 5A shows results of acute motor testing via a wire grip test. A control group, a single concussion group, and a repetitive concussion group were evaluated for wire grip score at various post-injury time points. Control, single, and repetitive mild TBI mice were assessed with a NSS at 1, 4, 24, 48, and 72 hours post-injury, as well as at 7 days and 1-month time-points. While a single mild TBI resulted in elevated NSS compared to controls (P<0.001), repetitive mild TBI mice exhibited significantly elevated scores compared to single mild TBI (P<0.001) and control (P<0.001) mice.

FIG. 5B shows results of acute motor testing based on a neurological severity score. A control group, a single concussion group, and a repetitive concussion group were evaluated for neurological severity scores at various post-injury time points. Mice underwent wire grip testing (WGT) 1 hour following TBI and on post-injury days 1-7. Repetitive mild TBI resulted in short-lived vestibulo-motor dysfunction compared to controls (P=0.01). Values are mean±SEM.

FIGS. 6A, 6B, 6C, and 6D show results of acute MWM acquisitions. A control group, a single concussion group, and a repetitive concussion group were evaluated for latency (in seconds) at 7 days, 14 days, 1 month, and 6 months, respectively. During acquisition training sessions, single mild TBI mice demonstrated an increased latency to find the platform compared to control mice acutely, at 2 weeks and at 1-month post-injury (P<0.001, P=0.01, and P=0.001, respectively) but was normalized compared to age-matched controls by 6 months (P=0.29). Repetitive mild TBI mice demonstrated a persistent, significant increase in escape latency acutely, at 2 weeks, at 1-month (P<0.001 at all time-points), as well as at 6-months post-injury (P=0.04).

FIGS. 6E, 6F, 6G, and 6H show results of acute MWM probe trials. A control group, a single concussion group, and a repetitive concussion group were evaluated for percentage of time spent in each of four quadrants (in order from left to right: southwest, northwest, northeast, and southeast). The platform was contained in the southwest quadrant. Evaluations were made at 7 days, 14 days, 1 month, and 6 months, respectively. During probe trials while control and single mild TBI mice demonstrated memory retention by spending significantly greater percentage of time in the target quadrant (SW) compared to all other quadrants, repetitive mild TBI mice spent a similar percentage of time in all quadrants, no greater than chance, and spent equivalent amount of time in the NW quadrant at 2 weeks (P=0.22); NW and NE quadrants at 1-month (P=0.93 and P=0.13, respectively); and NW quadrant at 6-months (P=0.84) compared to the target quadrant. (* P<0.05, ** P<0.01, *** P<0.001) Values are mean±SEM.

FIGS. 7A, 7B, and 7C, show results of delayed elevated plus maze trials. A control group, a single concussion group, and a repetitive concussion group were evaluated for total duration of time (in seconds) spent in open arms (left) and closed arms (right) of the elevated plus maze. Evaluations were made at 14 days, 1 month, and 6 months, respectively. Mild TBI results in reduced time spent on the open arms of the elevated plus maze (EPM) consistent with increased anxiety at 2 weeks post-injury (FIG. 7A). At 1-month post-injury, repetitive mild TBI mice spend more time on the open arms of the EPM compared to control mice consistent with decreased fear avoidance and increased risk-taking Single mild TBI mice were no different than controls (n.s., P=0.13) (FIG. 7B). By 6-months post-injury risk-taking behavior progressively increases in the repetitive mild TBI mice (FIG. 7C).

FIG. 7D shows results of a 1-month delayed Porsolt forced swim test. A control group, a single concussion group, and a repetitive concussion group were evaluated for total duration of immobility time (in seconds).

FIG. 7E shows results of a 1-month delayed tail-suspension test. A control group, a single concussion group, and a repetitive concussion group were evaluated for total duration of immobility time (in seconds).

FIG. 8A shows results of 1-month delayed sleep-wake behavior tests. A control group, a single concussion group, and a repetitive concussion group (from left to right) were evaluated for percentage time in each of wake, non-rapid eye movement (“NREM”) sleep, and rapid eye movement (“REM”) sleep. Mice with single or repetitive TBI exhibit a significant reduction in NREM sleep as well as a significant increase in wake time over the course of 24 hours. (* P<0.05, ** P<0.01, compared to control mice).

FIG. 8B shows results of 1-month delayed cortical activity tests during NREM sleep. A control group, a single concussion group, and a repetitive concussion group were evaluated for percentage prevalence of given frequencies of cortical activity. Quality of NREM sleep is disrupted by mild TBI. Power spectral analysis demonstrated a significant rightward “theta shift” in mild TBI animals. Control animals displayed significantly higher power spectrum for 1-2 Hz. Single mild TBI mice had significantly higher frequency at 4 Hz and repetitive mild TBI mice had significantly higher frequency at 5 Hz. (* P<0.05, ** P<0.01, *** P<0.001, control vs. single mild TBI; # P<0.05, ## P<0.01, ### P<0.001, control vs. repetitive mild TBI).

FIGS. 8C and 8D show results of 1-month delayed NREM sleep tests. A control group, a single concussion group, and a repetitive concussion group were evaluated for number of NREM episodes (FIG. 8C) and average duration of episodes (FIG. 8D). Repetitive mild TBI mice exhibited a greater number of NREM episodes compared to the single mild TBI and control groups, with significantly reduced episode lengths. Single mild TBI also resulted in shortened NREM segments. (* P<0.05, ** P<0.01) Values are mean±SEM.

FIGS. 9A, 9B, 9C, 9D, 9E, 9F, 9G, 9H, 9I, 9J, 9K, and 9L show mild TBI results in a dynamic astrocytic response. Representative GFAP immunostaining is shown for astrogliosis in the cortex, amygdala and dentate gyms of (FIGS. 9A-C) age-matched un-injured control, (FIGS. 9D-F) 6-month single mild TBI, and (FIGS. 9G-I) 6-month repetitive mild TBI mice. Single and repetitive mild TBI mice exhibited an acute elevation in GFAP staining in the cortex and amydgala at 7 days that resolved to control levels by 1-month (FIGS. 9J-K). GFAP was significantly decreased in the hippocampus of mild TBI mice compared to controls at 1-month. Repetitive mild TBI resulted in a second significant, diffuse astrocytic response at the 6-month timepoint, compared to control mice. (* P<0.05, ** P<0.01, *** P<0.001) Values are mean±SEM.

FIGS. 10A, 10B, 10C, 10D, 10E, 10F, 10G, 10H, 10I, 10J, 10K, and 10L show repetitive mild TBI results in a persistent diffuse increase in activated microglia. Representative CD68 immunostaining for activated microglia in the cortex, amygdala and dentate gyms of (FIGS. 10A-C) age-matched un-injured control, (FIGS. 10D-F) 6-month single mild TBI, and (FIGS. 10G-I) 6-month repetitive mild TBI mice. (FIGS. 10J-K) While mice receiving a single mTBI did not exhibit a significant elevation in activated microglia at any of the time-points, mice in the repetitive mTBI group had significantly increased microgliosis compared to single mild TBI and control mice at 7 days, as well as the 1- and 6-month time-points. (* P<0.05, ** P<0.01, *** P<0.001, repetitive mild TBI vs. control; # P<0.05, ## P<0.01, ### P<0.001, repetitive vs. single mild TBI) Values are mean±SEM.

FIGS. 11A, 11B, 11C, 11D, 11E, 11F, 11G, 11H, 11I, 11J, 11K, and 11L show repetitive mild TBI results in a persistent accumulation of phosphorylated tau. Representative AT8 immunostaining for phosphorylated tau in the cortex, amygdala and dentate gyms of (FIGS. 11A-C) age-matched un-injured control, (FIGS. 11D-F) 6-month single mild TBI, and (FIGS. 11G-I) 6-month repetitive mild TBI mice. (FIGS. 11J-K) Single and repetitive mild TBI mice exhibit significant increases in phosphorylated tau staining compared to age-matched, un-injured control mice at 7 days and 1-month. It was only in the repetitive mild TBI mice that the phosphorylated tau persisted at the 6-month time-point. (* P<0.05, ** P<0.01, *** P<0.001, repetitive mild TBI vs. control; # P<0.05, ## P<0.01, ### P<0.001, repetitive vs. single mild TBI) Values are mean±SEM.

FIGS. 12A and 12B show the dynamic pathophysiology of mild TBI. Generalized, global temporal pattern for astrogliosis, microgliosis and phosphorylated tau accumulation in (FIG. 12A) single mild TBI and (FIG. 12B) repetitive mild TBI mice. The slope of tau clearance in the single mild TBI mice is unknown, as is the overall pathophysiological course for both groups following 6 months (dashed lines).

FIGS. 13A, 13B, 13C, 13D, 13E, and 13F show astrocytosis, microgliosis and tauopathy in the CA1 and CA3 regions of the hippocampus. (* P<0.05, ** P<0.01, *** P<0.001, repetitive mild TBI vs. control; # P<0.05, ## P<0.01, ### P<0.001, repetitive vs. single mild TBI) Values are mean±SEM.

### DETAILED DESCRIPTION

There has been an increased interest in laboratory research focused on repetitive mild TBI. The behavioral deficits in these studies were more pronounced and protracted in the repetitive head injury groups; although, often these deficits improve or even resolve with time. A significant challenge for investigators developing experimental models of mTBI, is replicating the long-term neuropsychological and neurobehavioral consequences of mTBI such as deficits in memory acquisition and retrieval, attention and executive functioning, and reduced tolerance to stress. Contrary to some prior studies, the changes observed in repetitive mTBI mice progressed and even dynamically changed over a significant length of time.

Concussive head injuries have become a silent epidemic of increasing importance, with millions of sports-related concussions occurring each year. Recent evidence garnered from biophysics studies, advanced neuro-imaging findings, forensic analyses, and laboratory data has highlighted the ubiquity of concussion and subconcussion in contact sports and explosive blasts, as well as their potential to contribute to the development of sub-acute and chronic post-traumatic sequelae. Chronic traumatic encephalopathy (CTE) has become a popular topic due to its close association with a wide spectrum of sporting activities, including American football, hockey, soccer, boxing, and professional wrestling; as well as military personnel.

Most of our knowledge regarding CTE has come from post-mortem analyses and retrospective, demographic data. The precise incidence and prevalence of CTE is unknown and difficult to glean from sporadic case series. The risk factors for the development of CTE are not clear; however, the phenomenon seems to be caused by episodic and repetitive blunt force impacts to the head and transfer of acceleration-deceleration forces to the brain. Clinically, the disease presents as a composite syndrome of mood disorders, neuropsychiatric disturbance and cognitive impairment, with or without sensori-motor impairment. Definitive and confirmatory diagnoses of CTE still remain direct tissue histochemical and immunohistochemical analyses, which reveal topographically multifocal or diffuse cortical and subcortical hyperphosphorylated tauopathy; which is accompanied by isomorphic fibrillary astrogliosis, and microglial activation.

The underlying pathophysiological mechanisms in CTE have yet to be clearly elucidated. Some authors have cited a lack of prospective clinical evidence in associating repetitive TBI with CTE. Others have stated that the symptoms of CTE could be due to confounding factors such as co-morbid disease, medications, or normal aging. Generally, it takes years before the onset of symptoms of neurodegenerative disorders after an individual has experienced a TBI; and therefore, it requires an extremely long amount of time to gather this type of epidemiological data from the human population. Thus, an experimental animal model could truly help decipher the mechanisms by which CTE, as well as any of the other neurological sequelae such as post-concussion syndrome (PCS), post-traumatic stress disorder (PTSD), mild cognitive impairment (MCI), or dementia pugilistica (DP) may be triggered by repetitive brain injury.

Most experimental TBI also utilizes anesthesia during the delivery of repetitive injury, which confounds the pathophysiology and post-traumatic outcome. Experimental anesthetic exposure has been implicated in preconditioning, and has even been found to result in an increase in tau phosphorylation and cognitive impairment, particularly after repeated exposures. Few studies have attempted to perform injuries in un-anesthetized rodents. The injuries in these studies were delivered to hand-held rodents and were variable with regards to severity and outcome. The model of the subject technology is a non-anesthetized animal model of TBI that consistently and efficiently reproduces the spectrum of behavioral abnormalities and neuropathological findings observed in humans following mild TBI.

The adapted use of a controlled cortical impact (“CCI”) device does allow for precise control of the impact parameters, including velocity, duration and depth of impact; however, equally as important in developing this model was maintaining many of the principles of Marmarou's weight drop model of injury. The degree to which head motion is restricted is of chief importance in determining the relative contribution of impact and inertial components in head trauma. With the head free to move into a foam base of a known spring constant the shear forces between tissues are pre-dominant.

While most patients recover within a few days or weeks following a concussion, a number of individuals may develop long-lasting or progressively worsening symptoms, particularly if head injuries occur repetitively. In some patients, repetitive mild TBI may result in chronic neurodegenerative syndromes such as CTE. Models of the subject technology facilitate studies of closed head injury to investigate the neurological sequelae following repetitive mTBI. With models of the subject technology, animals exposed to a single mTBI have short-term “post-concussive” behavioral abnormalities. By contrast, the observed cumulative effect of 42 head impacts delivered over the course of seven days includes depression, risk-taking behavior, and persistent cognitive deficits.

This spectrum of behavior falls in line with emerging clinical data implicating repetitive head injury in the development of sub-acute and chronic neurological sequelae. Previous work using survey research from the Center for Study of Retired Athletes has shown that both MCI and depression are more common than expected in age-matched controls, and that there is a correlation for a higher incidence being associated with 3 or more concussions. In soccer, there has been the suggestion that repetitive heading of the ball may lead to an increased risk of chronic neurological injury. Studies in Division I college football players have shown that those experiencing three or more prior concussions had more symptoms and took longer to recover. In addition to cognitive impairment, patients sustaining mild TBI can endorse emotional symptoms, particularly when head impacts occur repetitively. Patients diagnosed with CTE have been noted to exhibit increased risk-taking and poor judgment. Depression is also common in post-concussive patients and is a prominent feature in many of those patients diagnosed with CTE.

Sleep disturbances can often occur acutely after a concussion and are a source of significant morbidity in patients with protracted post-concussive symptoms, particularly when head impacts occur repetitively. Mild TBI mice in this study spent more time awake and less time in NREM sleep over the course of 24 hours. Consistent with the overall reduction in sleep amounts, mild TBI caused more NREM sleep fragmentation. Repetitive mild TBI resulted in an increased number of short NREM segments. Taken along with the spectral analysis during NREM sleep, while control animals spent a significant portion of NREM time in deeper (stage 3/4) sleep, mild TBI mice spent more time in lighter (stage 1/2) stages of NREM sleep; failing to spend a lot of time in deeper, quality NREM sleep. It has been well documented that many of the patients that have been diagnosed with CTE may have difficulty falling asleep or staying asleep or may suffer from insomnia. Recent literature has also demonstrated that multiple head injuries are associated with an increased risk for and severity of sleep disturbance among active male military personnel, even when controlling for depression, post-traumatic stress disorder, and concussion symptom severity.

Most of our knowledge regarding CTE has come from post-mortem analyses and retrospective, demographic data. The risk factors implicated in the development of CTE following repetitive mild TBI, as well as the underlying pathophysiological mechanisms, have yet to be clearly elucidated and are difficult to discern from autopsy studies. Collectively, the spectrum of behavior observed in these mice, with this model of injury, recapitulates the neurobehavioral syndrome characteristic of CTE. Repetitive mild TBI in this model also resulted in the characteristic neuropathological features observed in patients diagnosed with CTE. Models of the subject technology allow for a controlled, mechanistic analysis of CTE, because it captures the spectrum of the human disease while not requiring the use of anesthesia.

The subject technology relates to an apparatus for the preparation of an experimental model for brain damage including neuronal and/or vascular impairments and evaluation for drug efficacy against brain impairments/damage, memory-related learning with magnetic resonance imaging (“MRI”), and a memory-related maze. According to some embodiments, implementations of the subject technology provide a device that reproducibly inflicts cerebral injuries in experimental animals.

According to some embodiments, implementations of the subject technology may be used to develop animal models of traumatic brain injury without the need for anesthesia in the animal. Animal models of repetitive concussion and subconcussion may be useful as an analog for human experience. According to some embodiments, implementations of the subject technology may be used to study long term neurodegenerative disease following repetitive traumatic brain injury, such as CTE. According to some embodiments, implementations of the subject technology provide high-throughput testing of pharmacological treatments for the treatment of concussion. According to some embodiments, implementations of the subject technology provide high-throughput testing of agents for the pharmacological prophylaxis of concussion and associated neurodegeneration.

In the following detailed description, numerous specific details are set forth to provide a full understanding of the subject technology. It will be apparent, however, to one ordinarily skilled in the art that the subject technology may be practiced without some of these specific details. In other instances, well-known structures and techniques have not been shown in detail so as not to obscure the subject technology.

Some traditional models of traumatic brain injury in animals use anesthesia to some degree (inhaled or intraperitoneal). Anesthesia is used in traditional models to restrain or subdue a subject for targeted impact. Anesthesia can be a confounding factor in traumatic brain injury experimentation. For example, anesthesia may cause hypothermia, neuroprotective responses, or other detrimental results.

Traditional models include CCI. This model allows for control over mechanical factors, such as velocity of impact and depth of resulting deformation, thus it offers potential advantages over the fluid percussion model, especially in biomechanical studies of TBI. The controlled impact is delivered to the intact dura by a compressed air-driven metallic piston causing deformation of the underlying cortex. Briefly, the device used in rodent CCI models consists of a pneumatic cylinder which is mounted on a cross bar so that the position of the impactor can be adjusted. The depth of cortical deformation is controlled by vertical adjustment of the crossbar holding the cylinder. It has been shown that cerebral hemodynamic responses such as elevated intracranial pressure, decreased blood and cerebral perfusion pressures, histological and cellular alterations, as well as functional deficits are related to both the depth of deformation and the velocity of the impact in CCI models. The pathobiology of CCI injury reproduces changes reported in clinical head injuries such as brain edema, elevated intracerebral pressure, reduced cortical perfusion, decreased cerebral blood flow, neuroendocrine and metabolic changes, and coma. Hence this model, which replicates clinical brain injury with skull deformation and related cortical compression, is extensively used to analyze complex molecular and genetic mechanisms underlying neuronal cell death and resulting neurological deficits following TBI. CCI offers the ability to control electronically the velocity, duration, and depth of the impact very precisely.

CCI commonly utilizes an anesthetized animal that has its head restrained in ear bars. As disclosed herein, anesthesia may be a confounding factor in TBI studies. While this model aids in targeting impact, it does not allow for the free movement of the head following impact from a pneumatic piston. This approach lacks clinical relevance as a model for traumatic impacts in which a subject's head would not be restrained. CCI models commonly involve drilling a hole in the skull (craniectomy) to impact the brain directly. This approach lacks clinical relevance as a model for traumatic impacts that are applied to a closed head.

Another traditional model is a weight drop model (i.e., Marmarou model). Marmarou's weight drop model is one of the most frequently used constrained rodent models of impact acceleration head injury. The trauma device consists of a column of brass weights falling freely by gravity from a designated height through a Plexiglas tube. After exposing the animal's skull by a midline incision, a stainless steel disc is rigidly fixed with dental cement to the animal's skull centrally between lambda and bregma fissures. The rats are then placed on a deep foam bed and the impact generated by dropping the brass weight onto the stainless steel disc. This method has been shown to produce graded brain injury in both rats and mice, where the injury severity is directly related to the mass and the height from which the brass weight is released. Morphological findings include an absence of supratentorial focal brain lesions. Histopathology shows widespread and bilateral damage of the neurons, axons, dendrites, and microvasculature. Reduced cerebral blood flow and elevated intracerebral pressure has been shown as a result of loss of cerebral autoregulation during the first 4 hours after the impact. Diffusion-weighted magnetic resonance imaging showed that there is a development of a vasogenic edema immediately after weight-drop impact followed by more widespread and slower edema formation due to a predominantly cellular swelling. Moreover, the Marmarou model has been shown to induce motor and cognitive deficits, similar to those shown after controlled cortical impact. This model induces both apoptotic and necrotic types of neuronal cell death.

Marmarou's weight drop model commonly relies on anesthesia to maintain the subject in one location. As disclosed herein, anesthesia may be a confounding factor in TBI studies. The weight drop model also commonly relies on surgery to implant a metal disc to the midline skull. Weight drop model can also be associated with high incidence of skull fracture. The severity of injury in Marmarou's weight drop model is based on the weight of the object dropped and height from which it is dropped.

According to some embodiments, disclosed herein are models of concussion/subconcussion that provide the benefits of precision targeting while allowing for the free movement of the head (e.g., into a foam pad). Approaches disclosed herein create acceleration/deceleration injury and more reliable axonal shearing than focal contusion.

According to some embodiments, models of concussion/subconcussion disclosed herein may be utilized without anesthesia. This mode of operation eliminates anesthesia as a confounding factor, thereby creating a more clinically relevant model for typical traumatic brain injury, in which the subject is not anesthetized. Accordingly, this mode of operation allows a user to explore more definitively the underlying pathophysiological response to trauma.

According to some embodiments, models of concussion/subconcussion disclosed herein may require no surgery. This mode of operation creates a more clinically relevant model for typical traumatic brain injury. This mode of operation also allows for easy modeling of repetitive injury over long periods of time because the subject does not require surgery and the associated recovery time or other avoidance of the effects of surgery.

According to some embodiments, models of concussion/subconcussion disclosed herein may be performed rapidly, for example in under 20 seconds. Such short procedure duration allows for high throughput animal testing.

According to some embodiments, models of concussion/subconcussion disclosed herein may include the use of a helmet to help prevent skull fractures. Use of a helmet also diffuses injury throughout the brain. This mode of operation creates a more clinically relevant model for traumatic brain injury types in which trauma and injury are diffused.

According to some embodiments, the subject technology includes a system and method for animal model concussion/traumatic brain injury and repetitive concussion/traumatic brain injury. The animal used in the model injury may be used as an analog for human traumatic brain injury. The animal used in the model may be any mammal. For example, animals contemplated may be rodents, including mice, rats, squirrels, porcupines, beavers, guinea pigs, and hamsters.

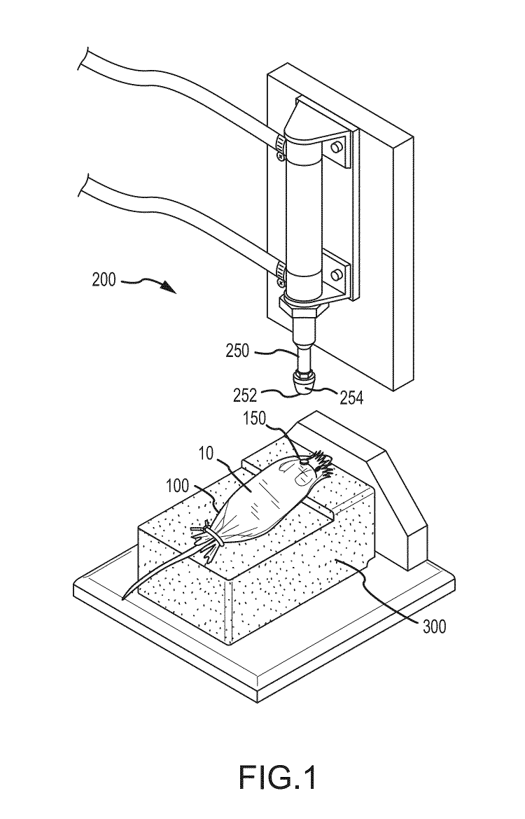

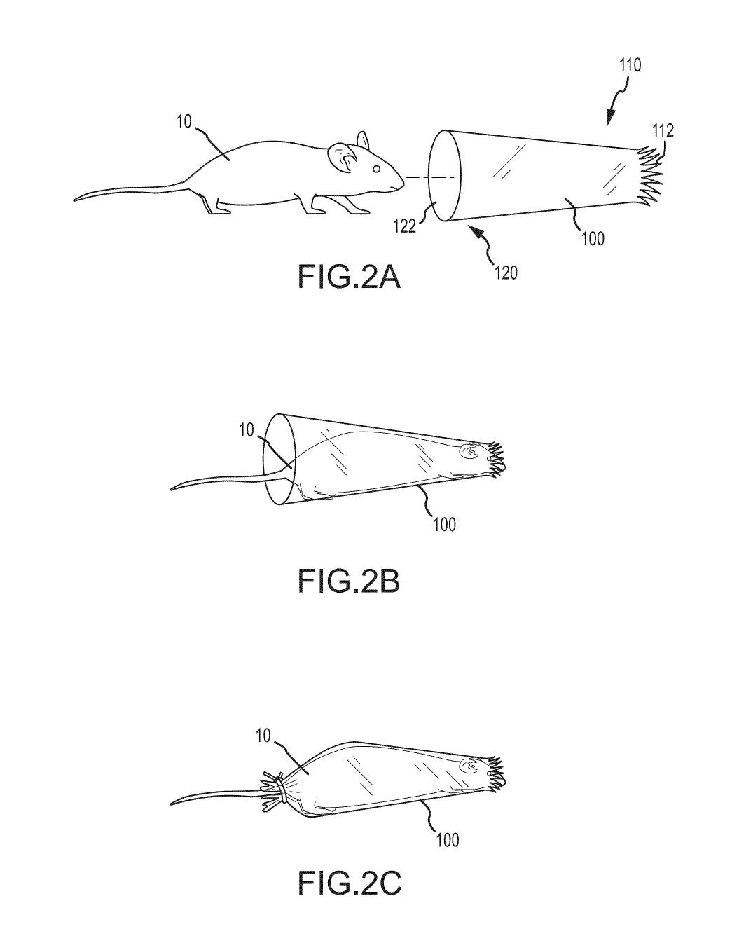

According to some embodiments, as shown in FIG. 1, an animal (e.g., a rodent) is placed into a containment sheath 100. The containment sheath 100 may be of a thin material shaped and sized to restrain the subject 10. The material may be somewhat flexible to allow movement of the head in response to an impact. However, the material may be sufficiently rigid to restrain the head within a range of motion that maintains an alignment with the impact device 200. The containment sheath 100 may be configured to restrain the limbs of the subject 10. For example, as shown in FIGS. 2A-2C, the containment sheath 100 may be substantially conical to receive and restrain a rodent. According to some embodiments, the containment sheath 100 may be configured to conform to both a head and a body of the subject 10, such that the head of the subject 10 is held stationary or substantially stationary relative to the body of the subject 10. By holding the head stationary, relative to the body, the subject 10 may be maintained in a position to allow precision targeting by an impact device 200. Alternatively, the containment sheath 100 may be configured to conform to only a body of the subject 10. According to some embodiments, the containment sheath 100 may be sized and shaped to receive any subject, including an animal subject, such as a mammal. The containment sheath 100 can be sized and shaped to encompass one or more limbs of the animal subject, such that the range of motion of the limbs is limited.

According to some embodiments, a back end 120 of the containment sheath 100 may have an opening 122 to receive the subject 10. As shown in FIG. 2C, the opening 122 may be closed by applying a tie posterior of the subject 10. By closing the opening 122, movement of the subject 10 posteriorly within the containment sheath 100 is limited or prevented. According to some embodiments, a front end 110 of the containment sheath 100 may have an opening 112 to provide an airway or other access for the subject 10.

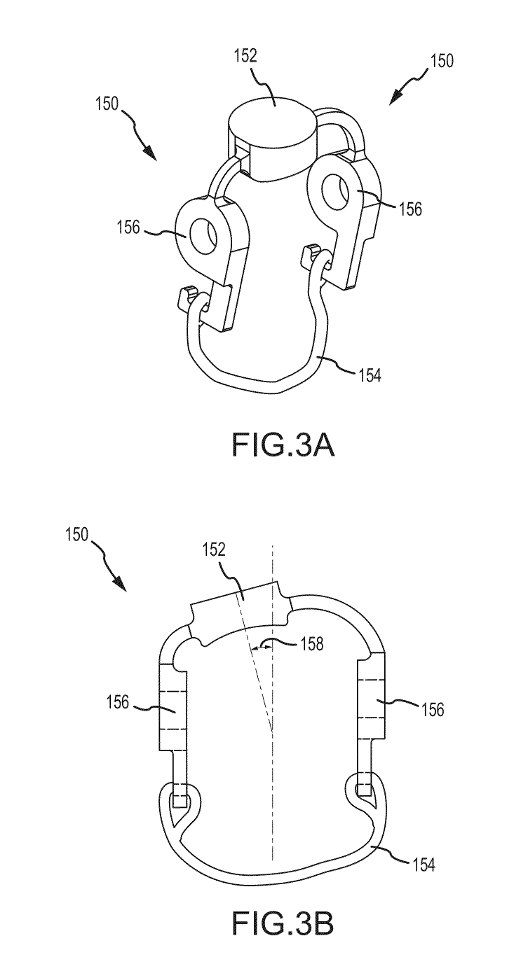

According to some embodiments, with the subject 10 resting in the containment sheath 100, a helmet 150 may be secured to the head. As shown in FIGS. 3A and 3B, The helmet 150 may include an impact plate 152 configured to receive an impact. The plate 152 can be positioned anywhere on the head. For example, the plate 152 may be centered over the left parieto-temporal cortex (between lambda and bregma). The plate 152 may be positioned by adjusting the entire helmet 150 relative to the head of the subject. The plate 152 may be positioned by having a predetermined location on the helmet 150. For example, as shown in FIG. 3B, the plate 152 may be eccentrically located between two handles 156. For example, the plate 152 may be eccentrically located along an arc spanning between two handles 156 of the helmet 150. An axis or surface of the plate 152 relative to one or more axes of the helmet 150 may form an angle 158 defining the location and orientation of the plate 152.

According to some embodiments, the plate 152 may have an inner surface configured to be placed against the head of the subject 10. The inner surface may conform to the contours of the head. The inner surface may include padding to insulate, cushion, or conform to the head of the subject 10. The padding may include a lining of, for example, 1.0 mm double-sided gel-tape. The inner surface and/or padding may simulate traumatic brain injury in the context of a human wearing a conforming helmet with a padded interior wall. For example, such a configuration could simulate traumatic brain injury inflicted upon athletes wearing helmets.

The helmet 150 may include a band 154 for securement around the head of the subject 10. The band 154 may be elastic or adjustable. The helmet 150 may be positioned outside the containment sheath 100 (i.e., upon an outer surface of the containment sheath 100). The helmet 150 may be positioned inside the containment sheath 100 directly upon the subject 10 (i.e., covered by an inner surface of the containment sheath 100).

According to embodiments, an impact device 200 is configured to apply a force to a subject 10 under controlled circumstances. The impact device 200 is configured to receive the subject 10. According to some embodiments, the subject 10 is positioned on a pad 300 of impact device 200. The pad 300 may be of a soft, conforming, or pliable material. For example, the pad 300 may be of foam. The pad 300 may be located beneath, across, or adjacent to at least a portion of an impact device 200, such as impactor 250. For example, the pad 300 may receive the head of the subject 10, such that the head is aligned in the pathway of an impactor 250. According to some embodiments, the head of the subject 10 is unbound relative to the pad 300, such that the head may move into the pad 300 in response to an impact. Prior to impact, the pad 300 provides stability to the head of the subject 10 as the subject is resting on the pad 300. The pad 300 also allows the head of the subject 10 to travel somewhat upon and after impact from the impactor 250. For example, the pad 300 may have a spring constant that allows the head of subject 10 to travel a given distance for a force applied to the head of the subject 10. In this regard, the pad 300 both stabilizes the head of the subject 10 while allowing travel and recoil of the head upon and after impact.

According to some embodiments, the subject 10 is subjected to an impact to simulate or induce a concussion or mild traumatic brain injury (mTBI). The impact may be delivered by an impact device 200 including the impactor 250. The impactor 250 may be a pressure-driven piston to advance from an initial position to a subsequent position impacting the head of the subject 10 with a programmable magnitude of force. In a pressure-driven system, a selectable magnitude of pressure may controllably determine the speed of and force applied by a piston. For example, a pneumatic cylinder may be used. Pneumatic cylinders use the stored potential energy of a fluid, such as compressed air, and convert it into kinetic energy as the air expands in an attempt to reach atmospheric pressure. This air expansion forces a piston to move in the desired direction at a desired velocity. The impactor 250 transfers the force it develops to the subject 10.

According to some embodiments, the impactor 250 can be mounted at any angle (e.g., relative to the subject 10 or a plane defined by the pad 300). For example, the impactor 250 may be aligned at an angle of 0°, ±10°, ±20°, ±30°, ±40°, ±50°, ±60°, ±70°, ±80°, ±90°, or greater with respect to a plane defined by the pad 300. The impactor 250 can be adjusted and aligned to be configured to impact the helmet plate 152 of the helmet 150.

Material composition of the impactor 250 and the diameter of the tip 252 of the impactor 250 contribute to the fracture threshold. The tip 252 of the impactor 250 may have a diameter of about 6 mm. For example, the tip 252 has a diameter of 1 mm, 2 mm, 3 mm, 4 mm, 5 mm, 6 mm, 7 mm, 8 mm, 9 mm, 10 mm, or greater. The tip 252 may include a cover 254 of a soft material. For example, the cover 254 may be of rubber or other material to reduce the likelihood of fracture upon impact.

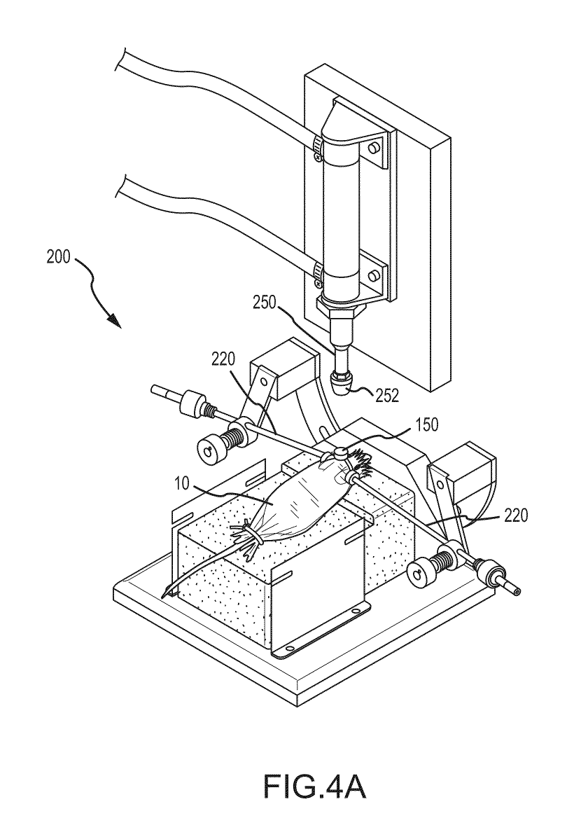

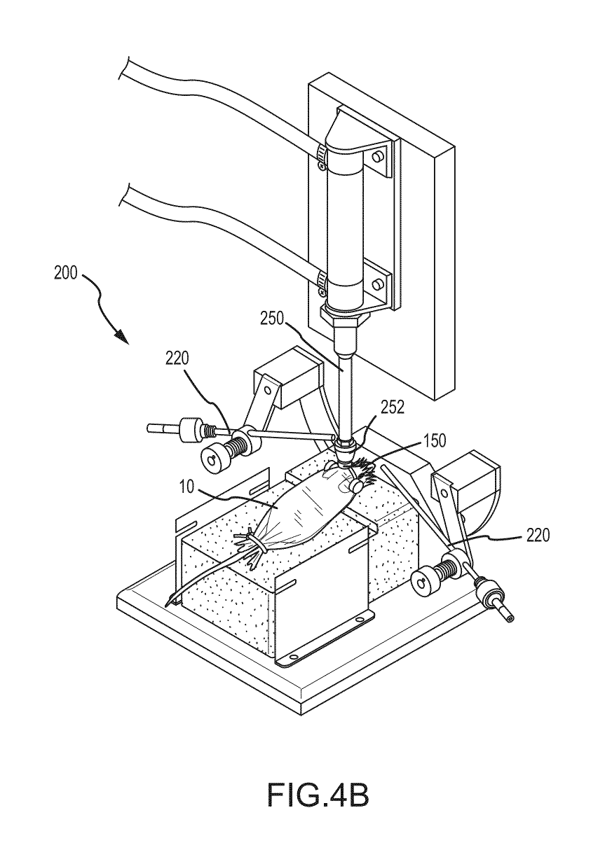

According to embodiments, as shown in FIGS. 4A and 4B, the impact device 200 may include at least one arm 220 that extends from a base 210 of the impact device 200 to connect to the helmet 150. According to embodiments, an arm 220 may releasably connect to one or more handles 156 of the helmet 150. For example, the arm 220 may magnetically connect to the helmet 150 at a handle 156.

According to embodiments, as shown in FIG. 4A, the arms 220 of the impact device 200 may connect to the helmet 150 prior to impact of an impactor 250. According to embodiments, the arms 220 secure at least a portion of a subject 10 prior to and leading up to impact by the impactor 250. This helps align and maintain alignment of the helmet 150 and the subject 10 relative to the impactor 250, such that targeting of an impact is precise and controlled. According to embodiments, the arms 220 may release from the helmet 150 prior to, at, or after a moment of impact (i.e., a moment of contact between the tip 252 and the head of the subject or the helmet plate 152). The strength of the magnetic connection of the arms 220 can be adjusted. Thus, if it is desired for the head to not move or for the head holder be utilized in prior models of head injury, this can allow for those models to be performed in awake animals. Various connection mechanisms between the arms 220 and the helmet 150 are contemplated, such as magnetic, frictional, electrostatic, etc.

According to embodiments, a location, orientation, and length of extension of an arm 220 may be set, adjusted, or modified relative to a base portion 210 of the impact device 200. Such configurations of an arm 220 correspond to at least a location or orientation of a helmet 150 relative to a base portion 210 of the impact device 200. Accordingly, at least a portion of a subject 10 may be controlled, with respect to its location and orientation relative to at least a portion of the impact device 200. For example, various articulating joints and/or extension capabilities may be provided. This allows a user to set and determine precise, controlled, and customizable targeting for impact on the subject 10.

According to embodiments, as shown in FIG. 4B, the arms 220 may be spring-loaded, such that advancement of the impactor 250 beyond a certain point along its path toward impact activates a release force that causes the arms 220 to disconnect from the handles 156. For example, the advancement of the impactor 250 may trigger a spring to apply a release force, greater than a connection force between an arm 220 and a handle 156, to release the helmet 150. Prior to advancement of the impactor 250, the release force may be prevented. For example, a spring or other mechanism may apply a release force prior to advancement of the impactor 250, wherein the release force is counteracted by a pin or other structure that maintains the arms 220 in a position for engaging the helmet 150. By further example, a spring or other mechanism may be prevented from applying a release force prior to advancement of the impactor 250, wherein the release force is applied upon removal of a pin or other structure that prevents the release force from being applied. According to embodiments, a connection force (e.g., magnetic force) between an arm 220 and a handle 156 is sufficient to restrain the subject 10 adequately. For example, the arms 220 are maintained in a consistent position, engaging the handles 156 to maintain the handles 156 in a consistent position. The connection force between the arms 220 and the handles 156 maintain the helmet 150 in a consistent position relative to the arms 220. According to embodiments, the release force is greater than the connection force. For example, the release force, when applied, overcomes the connection force to disengage the arms 220 from the helmet 150. According to embodiments, the arms 220 may move away from the helmet 150 by moving translationally or rotationally. For example, the arms 220 may rotate about an axis to disengage from the helmet 150. As shown in FIGS. 4A and 4B, the axis of rotation for the arms 220 may be an axis that does not intersect the helmet 150. According to embodiments, the motion of the arms 220 caused by the application of the release force may allow the arms 220 to rotate away from the helmet 150 with little or no combined torque being applied to the helmet 150. For example, as shown in FIGS. 4A and 4B, rotation of the arms 220 may be balanced such that any torque imparted upon helmet 150 by one of arms 220 is counterbalanced by a substantially equal and opposite torque applied by another of arms 220. This allows the helmet 150 to remain rotationally aligned relative to the impactor 250 (e.g., angle 158 is maintained). By further example, the arms 220 may retract away from the helmet 150 along a path that is linear, substantially linear, arcuate, or otherwise directs the arms 220 away from the helmet 150. According to embodiments, the motion of the arms 220 caused by the application of the release force may allow the arms 220 to move away from the helmet 150 with little or no combined force being applied to the helmet 150 in at least one axis. For example, as shown in FIGS. 4A and 4B, retraction of the arms 220 may be balanced such that any force imparted upon helmet 150 by one of the arms 220 in at least one axis is counterbalanced by a substantially equal and opposite force applied by another of the arms 220 in at least one axis. This allows the helmet 150 to remain locationally aligned relative to the impactor 250 in at least one axis.

According to embodiments, the subject 10 may be provided to the impact device 200 in a state that restricts free movement of the subject 10, as disclosed herein. According to embodiments, the sheath 100 may be used to restrain the subject 10 without use of the arms 220. According to embodiments, the arms 220 may be used to restrain the subject 10 without use of the sheath 100. According to embodiments, both the sheath 100 and the arms 220 may be used to restrain the subject 10.

According to embodiments, use of the impact device 200 does not require that the subject 10 be under the influence of anesthesia. According to some embodiments, systems and methods of the subject technology may be implemented while the subject 10 is anaesthetized. Accordingly, confounding factors associated with the effects of anesthesia in head trauma studies are reduced or eliminated. According to some embodiments, systems and methods of the subject technology may be implemented while the subject 10 is awake.

In addition, the magnetic head holder/stand is able to control and guide the injury location when the helmet is secured to the awake animal. The magnetic arms 220 attach to the helmet 150 and keep the head stable and secure. The arms 220 are adjustable and can be used to angle to head of the subject 10 to change the location of the impact. The arms 220 retract away from the helmet 150 upon impact allowing the head to move freely or to within a given range upon or after impact.

According to some embodiments, in preparation for impact, the tip 252 is lowered to touch the helmet 150. Thereby, the impact device 200 is calibrated with a “zero point” from which impact depth is measured. Having determined the zero point, the impactor 250 may retract. The tip 252 can then be driven from the retracted position to any depth beyond the zero point. For example, the tip 252 may be driven to a depth of 0.1 cm, 0.2 cm, 0.3 cm, 0.4 cm, 0.5 cm, 0.6 cm, 0.7 cm, 0.8 cm, 0.9 cm, 1.1 cm, 1.2 cm, 1.3 cm, 1.4 cm, 1.5 cm, 1.6 cm, 1.7 cm, 1.8 cm, 1.9 cm or greater beyond the zero point. The impact may cause a non-penetrating concussive blow.

According to some embodiments, the duration, force, and velocity of impact can be controlled or programmed. The duration of the impact may be 50 ms, 100 ms, 150 ms, 200 ms, or greater. The velocity may be 1 m/s, 2 m/s, 3 m/s, 4 m/s, 5 m/s, 6 m/s, 7 m/s, 8 m/s, 9 m/s, 10 m/s, or greater.

Various experimental models according to the subject technology can be used to evaluate drug efficacy of novel compounds against various cerebral impairments, especially for head injuries. Using experimental animals, novel chemical agents can be tested by administration to the animals before cerebral injuries by impact. Evaluation of improvements of cerebral injuries can also be monitored thereafter by comparison with non-treated animals. As an evaluation method, unless stated otherwise, MRI diagnosis is used. MRI evaluation can quantify damage by applying the imaging approach. The MRI approach can evaluate drug efficacy in a time-related fashion, and affords prevention and assessment of attenuation of TBI development, facilitating development of novel therapeutics for useful prevention and positive treatment of brain injuries. Reliability of evaluation is further enhanced when TBI is induced under specific conditions appropriate for generation of TBI in a reproducible manner. Furthermore, MRI diagnosis enables prompt monitoring of brain damage within about one hour after injury. In other words, animals yielding no or incomplete TBI can be readily identified and omitted from subsequent studies, saving time and unnecessary effort on something inappropriate for evaluation.

In head injuries, models disclosed herein may induce cerebral injuries affecting memory-related events after impact. Evaluation of brain injuries can be monitored by MRI diagnosis. Memory based on CA1 and CA3 fields of the hippocampus is affected as MRI portrayals revealed damage at the brain sites after impact.

Embodiments of the subject technology can be used for evaluation of drug efficacy of novel compounds on cerebral injuries or memory of animals. For example, when animals have been trained prior to head impact, the search-reward time-interval using the maze can be employed to test the drug effects on memory after head impact. If memory was intact, the search-reward time-interval would not be significantly abbreviated. However, if memory was affected, the time interval would be significantly extended. After a certain period has elapsed after head impact, animals are tested with the search-reward time-interval as the index of memory retention/loss. Such maze trials facilitate and contribute to evaluation of drug efficacy on memory-related events of animals subjected to cerebral injuries.

Furthermore, regardless of the use of the maze, MRI diagnosis of post-impact cerebral injuries to confirm generation of TBI is contemplated. By this confirmation, time and effort can be saved and reliability of drug evaluation on cerebral injuries can be enhanced. In addition in cases where histopathological evaluation has been done, studies on memory recovery of the animals cannot be assessed thereafter, although histopathological evaluation in combination with MRI diagnosis of post-impact cerebral injuries can to a certain extent measure memory outcome without anatomical procedures.

Embodiments of the present disclose provide testing systems and methods that provide (1) mechanical forces used to induce injury that are controlled, reproducible, and quantifiable; (2) inflicted injuries that are reproducible, quantifiable, and mimics components of human conditions; (3) injury outcomes, measured by morphological, physiological, biochemical, or behavioral parameters, that are related to the mechanical force causing the injury; and (4) programmable intensities of the mechanical force used to inflict injury that predict the outcome severity.

According to some embodiments, the subject 10 may be placed in a trial of a Morris water maze (“MWM”). In the typical MWM trial, a rat or mouse is placed into a small pool of water—back-end first to avoid stress, and facing the pool-side to avoid bias—which contains an escape platform hidden a few millimeters below the water surface. Visual cues, such as colored shapes, are placed around the pool in plain sight of the animal. The pool is usually 1.2 to 1.8 meter in diameter and 60 centimeters deep. The pool can also be half-filled with water to 30 centimeters in depth. A sidewall above the waterline prevents the rat or mouse from being distracted by laboratory activity, and from climbing out from the pool. When released, the subject swims around the pool in search of an exit while various parameters are recorded, including the time spent in each quadrant of the pool, the time taken to reach the platform (latency), and total distance traveled. Escape from the water reinforces a desire to find the platform quickly, and on subsequent trials (with the platform in the same position) subjects are able to locate the platform increasingly rapidly. This improvement in performance occurs presumably as a result of learning and memory for where the hidden platform is located relative to the conspicuous visual cues. After enough practice, a capable rat or mouse can swim directly from any release point to the platform.

There are a variety of MWM paradigms that can be used to examine different cognitive functions. In particular, cognitive flexibility can be assessed using a water maze paradigm in which the hidden platform is continually re-located. Various drugs can be applied to test subjects before, during, or after maze training, which can reveal information about physical ability. For example rats treated with the NMDA receptor blocker APV perform poorly in the Morris water maze, suggesting that NMDA receptors play a role in learning. And since long-term potentiation (“LTP”) also requires NMDA receptors, spatial learning may require LTP.

One measure of learning is latency, which is the time it takes to find the platform. However, rats and mice might develop search techniques that do not rely on spatial information, still getting to the platform relatively quickly. There are several analyzes that can tease out true spatial learning, many of which use probe trials during training: the escape platform is removed and the mice or rats are allowed to search for it for a fixed time (often 60 seconds). Analysis of the probe trial is made easier with a video tracker that traces the swimming patterns of the animals while searching for the platform. Commercial systems come with a suite of analysis features to extract measures such as time and path in quadrants, near platform, in any specified area. The Gallagher measure looks for average distance to platform. The Whishaw corridor test measures time and path in a strip from swim-start to platform.

According to some embodiments, other trials and testing methods may be employed. For example, a T-maze (or the variant Y-maze) may be used. A T-maze is a simple maze used in animal cognition experiments. It is shaped like the letter T (or Y), providing the subject, typically a rodent, with a straightforward choice. A test subject is placed at the base of the T. It must then decide whether to go left or right down either arm. Experimenters may place a reward in one arm of the maze, or different rewards may be placed in each arm. The subject may or may not be able to see what's at the end of the arm. In some cases, choosing to travel down one arm requires passing through a door which does not allow backtracking.

T-mazes can help researchers measure whether the test subjects have side preferences, alternate between choices, learn which side has a consistent outcome, or whether they have preferences between two options presented. Researchers have demonstrated that earthworms and other invertebrates are capable of having preferences in a T maze. A multiple T-maze is a complex maze made of many T-junctions.

According to some embodiments, an elevated plus maze trial may be employed. An elevated plus maze may be used to assess anxiety. The basic measure is the subject's preference for dark, enclosed places over bright, exposed places. The subject is placed in the center of the apparatus and observed for a set time. The model is based on rodents' aversion of open spaces. This aversion leads to the behavior termed thigmotaxis, which involves avoidance of open areas by confining movements to enclosed spaces or to the edges of a bounded space. In EPM this translates into a restriction of movement to the enclosed arms. Anxiety reduction in the plus-maze is indicated by an increase in the proportion of time spent in the open arms (time in open arms/total time in open or closed arms), and an increase in the proportion of entries into the open arms (entries into open arms/total entries into open or closed arms). Measurements compare the total time spent in the open arms (“OA”) and closed arms (“CA”) of the maze. Number of entries into the open and closed arms may also be measured. Total number of arm entries and number of closed-arm entries are usually employed as measures of general activity.

According to some embodiments, a grip strength assessment may be employed. The forelimb and/or hindlimb grip strength of a subject is tested using a specially designed measurement apparatus.

According to some embodiments, other trials and testing methods may include: fear conditioning, startle/ppi, radial arm maze, social interaction, ultrasonic vocalization, novel object recognition, conditioned taste aversion, conditioned place preference, aggression, open field, roto-rod, shirpa primary screen, light-dark box, social recognition, social transmission of food preference, vision penlight, hot plate, tail immersion, gait analysis, balance beam, forced swim, and tail suspension.

A neurological severity score may be given based on acute motor testing. Contributing factors for the score include a point for each failure to complete a task. The tasks include: (1) exit circle (ability and initiative to exit a circle of 30 cm diameter under a 3-minute time limit); (2) mono-/hemiparesis (paresis of upper and/or lower limb of the contralateral side); (3) straight walk (alertness, initiative, and motor ability to walk straight, once the mouse is put on the floor); (4) startle reflex (innate reflex; the mouse will bounce in response to a loud hand clap); (5) seeking behavior (physiological behavior as a sign of “interest” in the environment); (6) beam balancing (ability to balance on a beam of 7 mm width for at least 10 seconds); (7) round stick balancing (ability to balance on a round stick of 5 mm diameter for at least 10 seconds); (8) beam walk: 3 cm (ability to cross a 30 cm long beam of 3 cm width); (9) beam walk: 2 cm (same task, increased difficulty on a 2 cm wide beam); and (10) beam walk: 1 cm (idem, increased difficulty on a 1 cm wide beam). The highest possible score is 10 (failure for all tasks); the lowest possible score is 0 (success for all tasks).

### Example 1

Adult male, C57BL/6J mice (Jackson Laboratories, Bar Harbor, Me.) were purchased and housed with 5 mice per cage under standard laboratory conditions (automatically controlled temperature, humidity, ventilation, and 12 h light/dark cycle) with unlimited access to food and water throughout the study. Mice were allowed to adapt to the vivarium for at least 1 week prior to experimental procedures. After injury or neurobehavioral testing the animals were returned to their home cages.

A total of 280 mice were used for the study. At 12 weeks of age, mice undergoing injury were placed head first into a small plastic restraint bag/cone. Slits were cut at the narrow end of the cone to allow for increased head mobility and ventilation space. A twist tie was placed behind the mouse to immobilize the animal within the bag. A helmet was designed from 304 stainless steel, measuring 3 mm thickness and 6 mm diameter (Millenium Machinery, Rochester, N.Y.); which was secured to the head with an elastic band. The under-surface of the helmet was lined with 1.0 mm double-sided gel-tape. The helmet was engineered to fit the curvature of the mouse skull and cover the left hemisphere between lambda and bregma; up to but not crossing midline. Placing the anterior most part of the helmet 1-2 mm behind the left eye places the epicenter of the helmet over the left parieto-temporal cortex. The mice were then positioned on Type E foam padding (Foam to Size, Inc., Ashland, Va.) and positioned below the injury device.

Head impacts were delivered by a pressure-driven injury device. The impounder was rigidly mounted at a 20 degree angle from the vertical plane. In transitioning the model to a closed head injury, the impactor tip was modified to further reduce the incidence of skull fracture. The diameter of the tip was increased to 6 mm and constructed of softened rubber (Millenium Machinery, Rochester, N.Y.). To obtain a zero point, the impactor tip was carefully lowered until it touched the helmet surface. During impact the tip was driven pneumatically to a depth of 1 cm farther than the zero point. The duration of impact was 100 ms and was delivered at a velocity of 5 m/s. Following impact, the animals were then removed from the restraint bag and returned to their cage. Animals in the single mild TBI group received a single head impact, whereas mice in the repetitive mild TBI group received 6 head impacts per day (each hit separated by 2 hours) for 7 days straight—for a total of 42 head impacts. Sham-injured/control animals underwent the identical procedure as the trauma groups; however, no injury is delivered. All control mice were age-matched, to account for any age-related differences in behavior or pathology. Mice were then assessed in a blinded fashion using various neuro-behavioral tests, as described below. In addition, all mice were used only once for an individual behavioral task at any time-point.

To broadly characterize the effects of a single and repetitive mild TBI in this model, a neurological severity score was obtained to evaluate the neurological impairment compared to un-injured controls. The NSS is a composite clinical score consisting of 10 individual clinical parameters, including tasks on motor function, alertness and general physiological behavior (Supplementary Table 1). Mice (10 per group) were tested at 1, 4, 24, 48, and 72 hours post-injury, as well as at 7 days and 1-month time-points. The severity of injury is defined by the initial NSS measured at 1 hour post-TBI, and is a reliable predictor of late outcome.

Vestibulo-motor function was assessed by wire grip testing 1 hour following TBI and on post-injury days 1-7, as previously described. Briefly, mice (10 per group) were picked up by the tail and placed on a taut metal wire suspended between 2 upright bars 50 cm above a padded table. The time and manner in which the mouse could hold onto the wire were noted and scored on a scale of 0-5. Mice were tested 3 consecutive times at each time-point. The score reported is the average of these individual trials. A composite group score was then calculated as the mean of these scores at each time-point and then used for analysis.

Control, single mild TBI, and repetitive mild TBI groups were tested in the Morris water maze (MWM) acutely (starting 24 hours following last TBI), sub-acutely (starting post-injury day 9), and chronically at 1 month and 6 months following their last head impact. Briefly, mice had to locate an invisible platform submerged 5 mm below the water level in a circular pool (dimensions 120×30 cm, temperature 22±1° C.), based on the spatial location of 5 strategic visual cues fixed at distinct positions around the pool, as described previously. The water was made opaque by adding nontoxic, water-soluble Tempera paint (Sargent Art, Inc., Hazleton, Pa.). Data were recorded with the help of video cameras (Sony ExWave HAD) with a computerized video tracking system (Biobserve Viewer3 software, St. Augustin, Germany).

For training days (acquisition phase), all mice (14-15 per group, per time-point) were given a maximum test duration of 60 seconds to locate the hidden platform. The latency to reach the platform was recorded by the computerized video tracking system. Mice that failed to locate the platform within the time limit were guided to it and allowed to rest and orient themselves for 15 s. The acquisition phase testing was conducted over five consecutive days, with four trials on each day, with the goal of locating the submerged hidden platform from different starting points/orientations (north, south, east, west). On Day 6 of MWM testing, all animals were tested for visual acuity and swimming speed using a visible platform paradigm. None of the animals were excluded from further testing based on the visual acuity and motor evaluation tests. Day 6 all mice also underwent a probe trial (retention phase) where the platform was removed from the pool. Mice were given 30 seconds to swim and time spent in the target quadrant (quadrant where the platform had been) versus the other quadrants was assessed, as described previously.

Anxiety-related and risk-taking behavior of the mice were evaluated using the elevated plus maze test. When mice are placed in an elevated plus maze apparatus, both exploratory and fear drives are evoked, with rodents displaying a natural aversion for the open arms of the maze. Mice (15 per group, per time-point) were evaluated at 2 weeks, 1 month, and 6 months from the last head impact. The EPM consisted of two opposing open arms (35 cm×5 cm) and two closed arms (35 cm×5 cm×15 cm) that extended from a central platform (5 cm×5 cm) elevated 60 cm above the floor. A small raised lip (0.5 cm) around the edges of the open arms prevents animals from slipping off. Mice were placed individually on the central platform facing an open arm, away from the examiner and were allowed to freely explore the maze for 5 min under even overhead fluorescent lighting, as described previously. The behavior of each mouse was monitored using a video camera (Sony ExWave HAD) and the movement of the mice automatically registered and analyzed with a computerized tracking system (Biobserve Viewer3 software, St. Augustin, Germany). Time spent in the open and closed arms was measured and each mouse was only tested once in the maze. The floor of the EPM was washed after each testing with 0.1% acetic acid solution to remove odors left by previous subjects.

To determine the long-term effects of mild TBI on depression-like behavior, mice (15 per group) were tested in the Porsolt forced swim test and the tail suspension test at 1 month post-injury, as previously described.

Porsolt forced swim test: Briefly, mice were placed in an open glass cylinder (diameter 12 cm, height 24 cm, water level 16 cm) containing fresh tap water of 23-25° C. The duration of the entire test was 6 minutes; with the first 2 minutes used for habituation and only the last 4 minutes used for analysis. Two different experimenters blinded to the groups of the mice evaluated the behavior of the animals manually. A mouse was judged to be immobile when it remained floating in the water, making only those movements necessary to keep its head above water surface.

Tail suspension test: Briefly, mice were suspended by the tail to a bar elevated 40 cm above the surface of a table covered with soft cloth in a sound-proof room. The duration of the test was 6 minutes. Two different experimenters blinded to the groups of the mice evaluated the behavior of the animals manually. The immobility time of the tail-suspended mice was measured and defined as the absence of limb movement.

Surgical implantation of Electroencephalography (“EEG”) and Electromyography (“EMG”) electrodes: Electroencephalography and EMG data was acquired in mice at 1 month post-injury (8-9 per group) using implantable telemetry devices (Data Science International, ST. Paul, Minn., USA) and Dataquest A.R.T. system. As the F20-EET EEG telemetry device is one of the smallest commercially available devices (3.9 grams), it allowed for recording of EEG and EMG in freely moving animals. Surgery was done under anesthesia (ketamine-xyalzine) 24 hours before recording. The body of the transmitter was implanted intra-peritoneally via a midline abdominal incision. Electroencephalography lead implantation was performed via insertion of leads in small burr holes overlying the cortex. Electromyography leads EMG electrodes were made from multi-stranded stainless steel wires and were implanted into the neck muscle. The EEG electrodes were secured with dental cement and the EMG electrodes were secured with sutures. Mice were allowed to recover and were individually housed in a sound-attenuated and ventilated chamber on a standard light-dark cycle, with food and water available ad libitum.

Data Acquisition and Analysis: Following a one-day acclimation and recovery period, the telemetry EEG/EMG devices were activated and continuous recording were obtained for a total of 24 hour (6 pm-6 pm), as previously described. The EEG data was analyzed in 1-minute epochs using the Neuroscore software devices (Data Science International, St. Paul, Minn., USA). Using both manual scoring as well as automated software, EEG/EMG recordings were broken down into active wake, NREM sleep, and REM sleep. Active wake was classified as low-amplitude EEG with high EMG activity. NREM sleep was classified as high-amplitude EEG dominated by delta band components (0-4 Hz). REM sleep was classified as low amplitude EEG with low EMG activity. In addition to total sleep/wake time, power band (i.e. delta, theta, alpha, beta) and power spectral (frequency) analysis of sleep/wake states were further assessed to study the quality of NREM and REM sleep in each animal.

All data is presented as the means±standard error of the mean (“SEM”). Analysis of variance for repeated measures was used to determine statistically significant differences for NSS, wire grip test, and acquisition trials of the MWM. All other behavioral data comparisons were analyzed with a one-way analysis of variance (“ANOVA”) followed by Student-Newman-Keuls (“SNK”) or Tukey post-hoc test, using the StatSoft Statistica 7 program. Differences were considered statistically significant if P<0.05.

In models of the subject technology, the injury can be inflicted without the use of anesthesia. The animals were immobilized with a commercial plastic cone/bag restrainer normally used for tail vein injections. The mouse is placed into the bag headfirst and secured behind with a bag tie. In this manner the animal is comfortably swaddled—obviating the need for anesthesia (FIGS. 1-2C). The mouse is placed on a foam base, to allow for the acceleration-deceleration component of injury key to human concussive injury. The injuries are delivered with a CCI device; however, to transition the injury to a closed-head model the metal impact tip was replaced with a larger diameter rubber tip and developed a protective mouse helmet (FIGS. 3A-B). This ultimately allows for accurate delivery and diffuse spread of the force of the impacts. The impacts are administered to left side of the “helmeted” mouse head, with the entire injury process reliably performed in less than one minute. There were no post-traumatic apneic episodes or seizure activity observed, and no mortalities secondary to any of the impacts.

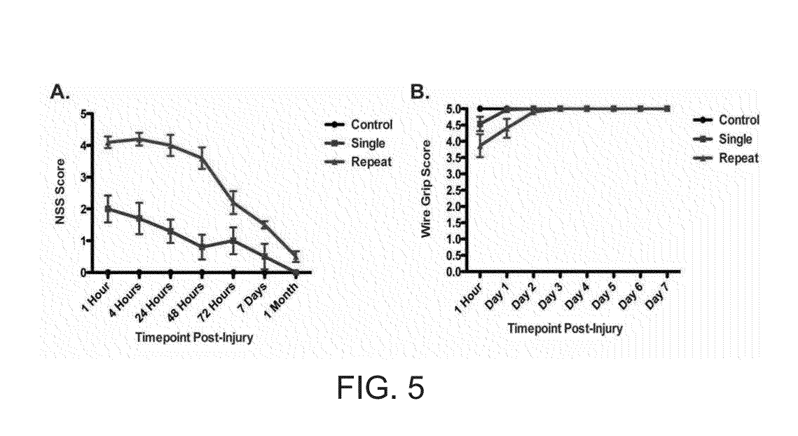

In NSS testing, the severity of impact for both the single and repetitive TBI groups fell in the “mild” spectrum. There were significant main effects of injury group (F_{(2,27)}=65.2, P<0.001; ANOVA), with NSS significantly increased in mice receiving repetitive TBI compared to single TBI (P<0.001) and control (P<0.001) groups (FIG. 5A). Single TBI mice had significantly elevated NSS compared to un-injured controls as well (P<0.001). Disruption of static and dynamic balance has also been thoroughly described in patients with concussion. Vestibulo-motor function was assessed by wire grip testing and there were significant main effects of injury group (F_{(2,27)}=4.80, P=0.02; repeated measures ANOVA) on performance. While a single TBI did not result in significant deficits on wire grip testing (P=0.08), the repetitive TBI group demonstrated deficits compared to control mice (P=0.01), which resolved within 48 hours (FIG. 5B).

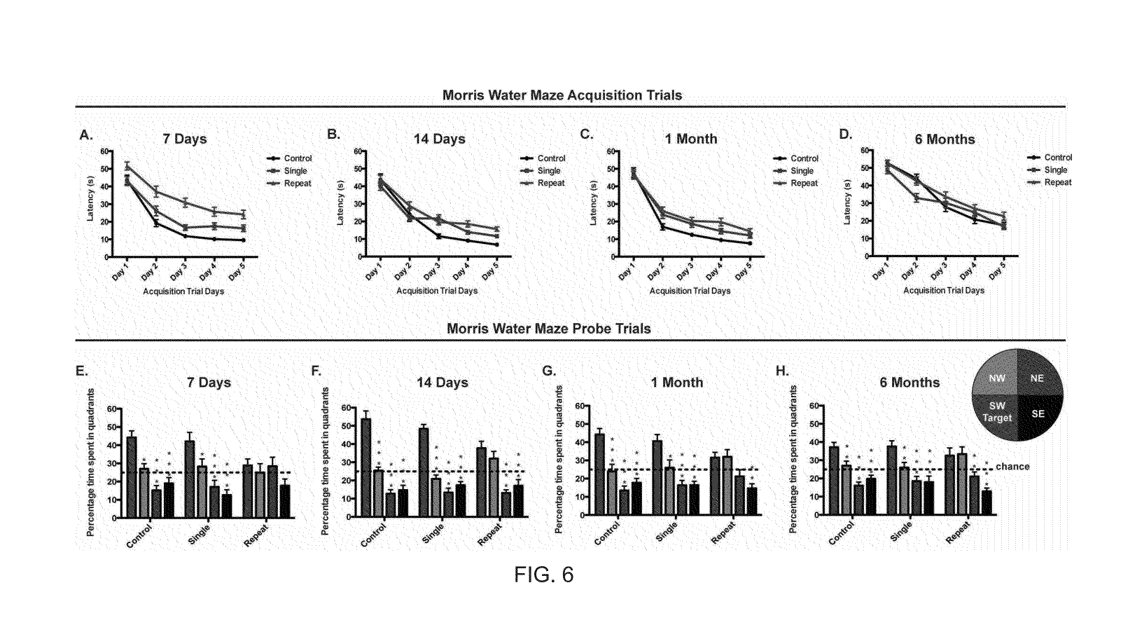

Next, the hippocampus-dependent spatial learning and long-term memory were assessed in the single and repetitive TBI mice using the Morris water maze. Animals from all groups showed daily improvements in their abilities to locate the hidden platform during the acquisition phase of the MWM task; however, mild TBI mice showed an impaired learning profile. There were significant main effects of injury group acutely (F_{(2,165)}=36.27, P<0.001; repeated measures ANOVA), at 2 weeks (F_{(2,197)}=9.77, P<0.001), 1 month (F_{(2,161)}=10.57, P<0.001), and 6 months (F_{(2,170)}=4.86, P=0.008). During acquisition training sessions, repetitive mild TBI mice demonstrated a persistent, significant increase in escape latency out to 6 months (FIG. 6A-D). Findings from the probe test indicate that mice from the single mild TBI and un-injured control groups, at all time-points, spent a significantly higher percentage of time in the target quadrant (the location that contained the platform during training) when compared to the other equivalent zones (FIG. 6E-H). In contrast, repetitive mild TBI mice exhibited impaired spatial memory, failing to show significant discrimination and preference for the target quadrant, compared to the other quadrants, acutely through the 6-month time-point (FIG. 6E-H).

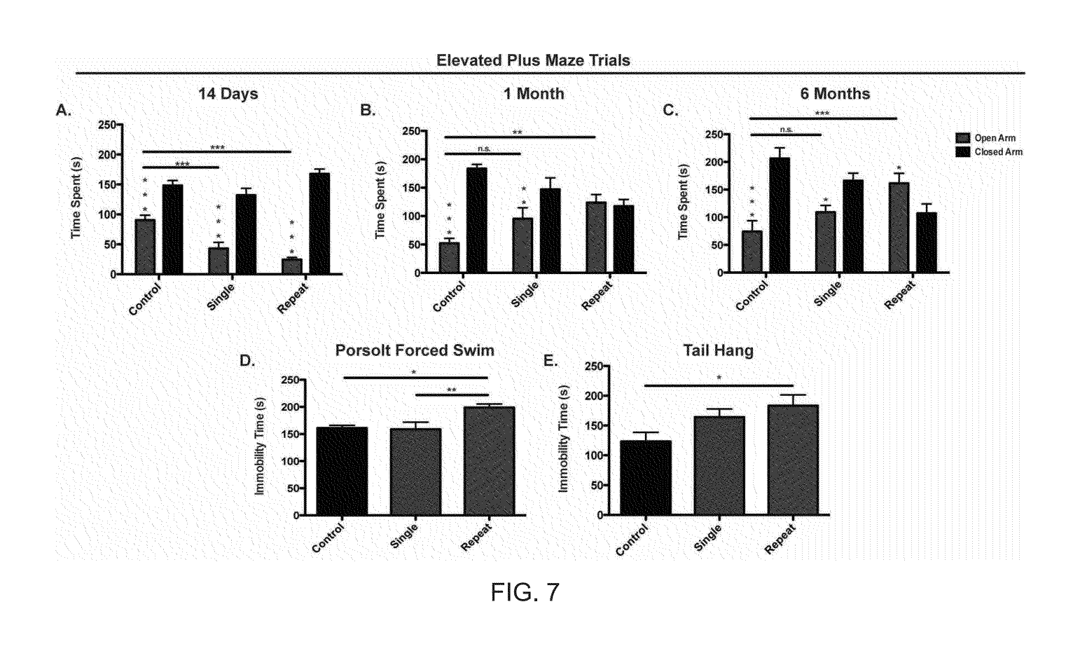

The elevated plus maze was used to characterize the effect of single and repetitive mild TBI on anxiety-related and risk-taking behaviors. At 2 weeks post-injury, mild TBI mice exhibited increased anxiety-like behavior (FIG. 7A). All mice spent significantly less time in the open arms than the closed arms (all groups P<0.001). However, single and repetitive mild TBI resulted in significantly reduced time spent in the open arms of the maze compared to control mice (P=0.004 and P<0.001, respectively), consistent with increased anxiety. At the 1- and 6-month time-points control mice spent significantly less time in the open arms compared to the closed arms (P<0.001, both time-points), as did the single mild TBI mice (P=0.009 and P=0.01, respectively) (FIG. 7B,C). While there was no difference in the amount of time spent on the open arms between single mild TBI and control mice at 1 and 6 months (P=0.13 and P=0.18, respectively); at the 1-month time-point, mice in the repetitive mild TBI group spent an increased amount of time on the open arms of the EPM compared to control mice (P=0.008)—interestingly spending nearly an equivalent amount of time on the open arms as the closed arms (P=0.78) (FIG. 7B). Such increased exploratory activity in the open arms and reduced fearfulness is consistent with increased risk-taking as noted in other studies. This increased risk-taking persisted and progressed in the repetitive mild TBI mice out to 6 months, with mice spending significantly greater time in the open arms compared to controls (P<0.001); and at this point even spending significantly greater time on the open arms than on the closed arms (P=0.02) (FIG. 7C).

At 1 month post-injury, in the Porsolt forced swim test, there was a significant effect of injury severity on depression-like behavior; with significantly increased immobility time in the repetitive mTBI group compared to the single mild TBI and control groups (P=0.009 and P=0.01, respectively) (FIG. 7D). Single mild TBI mice did not exhibit depression-like behavior. The tail-suspension test revealed comparable effects of repetitive mild TBI on depressed behavior as the forced swim test. Repetitive mild TBI mice demonstrated significantly increased immobility time compared to the control group (P=0.03) (FIG. 7E). Again as with the forced swim test, there was no effect of a single mild TBI on immobility time in the tail suspension test at 1 month post-injury.

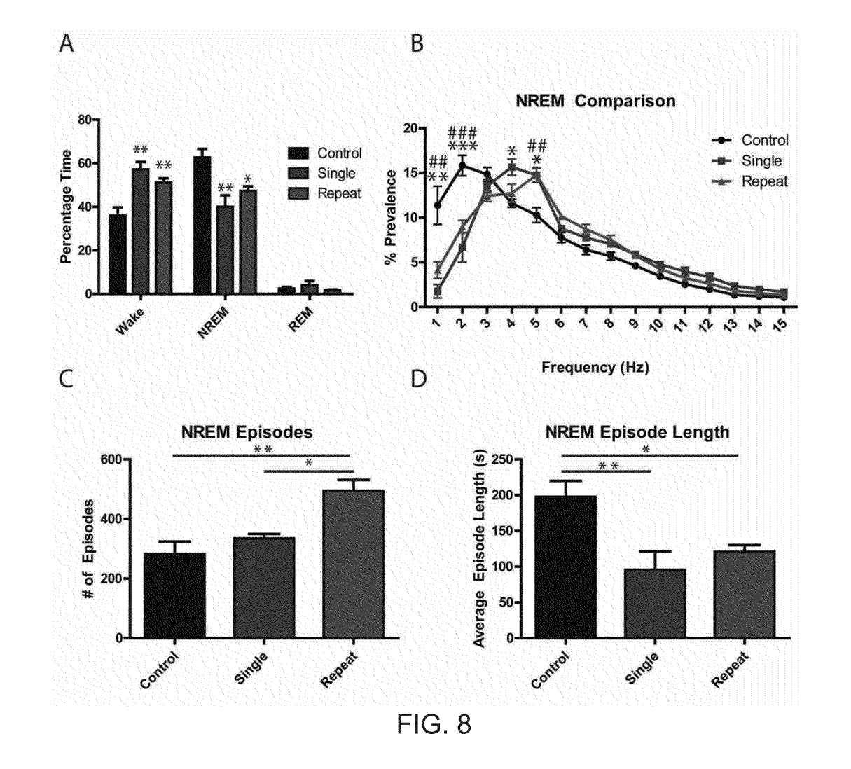

To evaluate the long-term effect of mild TBI on sleep-wake behavior, infrared videography and electrophysiological monitoring were used. At 1 month post-injury, mice with single or repetitive mild TBI exhibited a significant increase in wake time (P=0.001 and P=0.005, respectively) and a concomitant reduction in NREM sleep (P=0.002 and P=0.02, respectively) (FIG. 8A). The quality of NREM and REM sleep were examined in these mice. During NREM sleep there was an increase in cortical activity with a significant shift towards higher frequencies (FIG. 8B). Control mice had significantly higher frequencies compared to single and repetitive TBI mice at 1 Hz (P=0.005 and P=0.009, respectively) and 2 Hz (P=<0.001, both groups). Single mild TBI mice had significantly higher frequency at 4 Hz (P=0.02) and 5 Hz (P=0.01); and repetitive mild TBI mice had significantly higher frequency at 5 Hz (P=0.003) compared to control mice. Mild TBI caused more NREM sleep fragmentation as well. The number of NREM episodes was significantly increased in the repetitive mild TBI mice compared to single mild TBI (P=0.04) and un-injured control mice (P=0.002) (FIG. 8C). At the same time, single and repetitive mild TBI caused a significant decrease in the length of each NREM episode (P=0.009 and P=0.02, respectively) (FIG. 8D). Mild TBI did not result in a significant difference in the quality of REM sleep compared to control animals (data not shown).

### Example 2

The previously described model allows for controlled, closed-head impacts to un-anesthetized mice. Briefly, 12-week old mice were divided into three groups: control, single, and repetitive mild TBI. Repetitive mild TBI mice received six concussive impacts daily, for seven days. Mice were then subsequently sacrificed for macro- and micro-histopathologic analysis at 7 days, 1-month and 6-months after the last TBI received. Brain sections were immune-stained for glial fibrillary acidic protein(GFAP) for astrocytes, CD68 for activated microglia, and AT8 for phosphorylated Tau protein.

Brains from single and repetitive mild TBI mice lacked macroscopic tissue damage (contusion, necrosis, or hemorrhage) at all time-points. Single mild TBI resulted in an acute reactive astrocytosis at 7 days and phosphorylated tau deposition that is present acutely and at 1-month but not persistent out to 6-months. Repetitive mTBI resulted in a more marked neuro-inflammatory response, with persistent and widespread astrogliosis and microglial activation, as well as accumulation of phosphorylated tau protein out to 6-months.

The neuropathological findings in this model of repetitive mild TBI resemble the histopathological hallmarks of CTE, including increased astrogliosis, microglial activation, and hyperphosphorylated tau protein accumulation.

A model of closed-head injury in un-anesthetized mice can encapsulate the neuro-behavioral spectrum observed in CTE patients, including cognitive deficits, increased risk-taking, depression and sleep disturbances. We hypothesized that repetitive mild TBI in this model would result in the histopathological hallmarks of CTE, including increased astrogliosis, microglial activation, and hyperphosphorylated tau protein accumulation.

All animals used in this study were treated in accordance with the National Institute of Health Guidelines for the Care and Use of Laboratory Animals. Adult male, C57BL/6J mice (Jackson Laboratories, Bar Harbor, Me.) were purchased and housed with 5 mice per cage under standard laboratory conditions (automatically controlled temperature, humidity, ventilation, and 12 h light/dark cycle) with unlimited access to food and water throughout the study. Mice were allowed to adapt to the vivarium for at least 1 week prior to experimental procedures. After injury or neurobehavioral testing, the animals were returned to their home cages.

A total of 280 mice were used for the study. At 12 weeks of age, mice undergoing injury were placed head first into a small plastic restraint bag/cone. Slits were cut at the narrow end of the cone to allow for increased head mobility and ventilation space. A twist tie was placed behind the mouse to immobilize the animal within the bag. A helmet was designed from 304 stainless steel, measuring 3 mm thickness and 6 mm diameter (Millenium Machinery, Rochester, N.Y.); which was secured to the head with an elastic band. The under-surface of the helmet was lined with 1.0 mm double-sided gel-tape. The helmet was engineered to fit the curvature of the mouse skull and cover the left hemisphere between lambda and bregma; up to but not crossing midline. Placing the anterior most part of the helmet 1-2 mm behind the left eye places the epicenter of the helmet over the left parieto-temporal cortex. The mice were then positioned on Type E foam padding (Foam to Size, Inc., Ashland, Va.) and positioned below the injury device.

Head impacts were delivered by a pressure-driven injury device that is mechanically identical to our previously described controlled cortical impact device. The impounder was rigidly mounted at a 20 degree angle from the vertical plane. In transitioning the model to a closed head injury, the impactor tip was modified to further reduce the incidence of skull fracture. The diameter of the tip was increased to 6 mm and constructed of softened rubber (Millenium Machinery, Rochester, N.Y.). To obtain a zero point, the impactor tip was carefully lowered until it touched the helmet surface. During impact the tip was driven pneumatically to a depth of 1 cm farther than the zero point. The duration of impact was 100 ms and was delivered at a velocity of 5 m/s. Following impact, the animals were then removed from the restraint bag and returned to their cage. Animals in the single mild TBI group received a single head impact, whereas mice in the repetitive mild TBI group received 6 head impacts per day (each hit separated by 2 hours) for 7 days straight—for a total of 42 head impacts. Sham-injured/control animals underwent the identical procedure as the trauma groups; however, no injury is delivered. All control mice were age-matched, to account for any age-related differences in pathology.

Age-matched controls and mice 7 days, 1 month, or 6 months following single or repeated mTBI underwent transcardial perfusion with ice-cold heparinized 0.01M phosphate-buffered saline (PBS) (pH 7.4, Sigma-Aldrich), followed by fixation with 4% para-formaldehyde (PFA) (Sigma-Aldrich) in PBS. Cerebral tissue from all animals was dissected from the calvarium and post-fixed in 4% PFA for 24 hours. Following fixation, cerebral tissue underwent graduated dehydration first in 15% and then 30% sucrose (Sigma-Aldrich) for 24 hours each. Dehydrated tissue was placed in Optimal Cutting Temperature (O.C.T.) Compound (Tissue-Tek) and was sliced on a calibrated cryostat (Leica CM1900) into 30 μm sections. Tissue sections were then floated in PBS.

The primary antibodies used included rabbit anti-mouse glial fibrillary acidic protein (GFAP) polyclonal IgG (AB5804, Millipore), mouse anti-human phospho-PHF-tau (pTau) monoclonal IgG (specific for pSer202/pThr205 tau phosphorylation sites) (MN1020, Thermo Scientific), and rat anti-mouse CD-68 (macrosialin) (MCA1957, Serotec) diluted to 1:1000, 1:100, and 1:250, respectively. The secondary antibodies used were all diluted to 1:250 and included donkey anti-rabbit Cy2 conjugated IgG (711-225-152, Jackson ImmunoResearch), donkey anti-mouse Cy3 conjugated IgG (715-165-150, Jackson ImmunoResearch), and donkey anti-rat DyLight488 conjugated IgG (712-485-150, Jackson ImmunoResearch). All sections were blocked with 0.5% Triton X-100 (Acros Organics) in 0.01M phosphate-buffered saline (PBS) (pH 7.4, Sigma-Aldrich) and 7% normal donkey serum (NDS) (017-000-121, Jackson ImmunoResearch). Primary and secondary agents were diluted in 0.1% Triton X-100/PBS and 1% NDS. Secondary antibodies alone served as negative controls. Four mice per group were used for staining procedures. All sections were mounted with Prolong Antifade Gold with 4′,6-diamidino-2-phenylindole (DAPI) (Invitrogen) as a nuclear counter stain.

Antibodies were detected at the appropriate wavelength at a magnification of 40× on confocal microscopy (Olympus IX81, Fluoview v. 4.3), using a standard laser power, image PMT, and gain. In each slice collected, three evenly distributed images at a uniform depth from the surface of the dorsal cortex, as well as one CA1, CA3, dentate gyms (DG), and amygdala image was obtained in each cerebral hemisphere. All images were acquired at a resolution of 1024×10²⁴ pixels. All image acquisition was performed blinded to experimental group.

All image analysis was performed blinded to experimental group. ImageJ software was used to apply a standard threshold to all immunohistochemical images. The pixel area encompassed by this thresholding scheme was then quantified and normalized relative to control pixel areas.

All data is presented as the means±standard error of the mean (SEM). Statistical analysis was performed using the StatSoft Statistica 7 program. Normalized GFAP, pTau, and CD-68 immunoreactive areas were evaluated using a one-way analysis of variance (ANOVA) with an all pair wise multiple comparison procedure (Tukey post-hoc method). Differences were considered statistically significant if P<0.05.

Gross examination of the brains in the single and repetitive mTBI groups did not reveal any evidence of brain atrophy, tissue loss, hemorrhage (subdural, epidural, subarachnoid), or contusion.

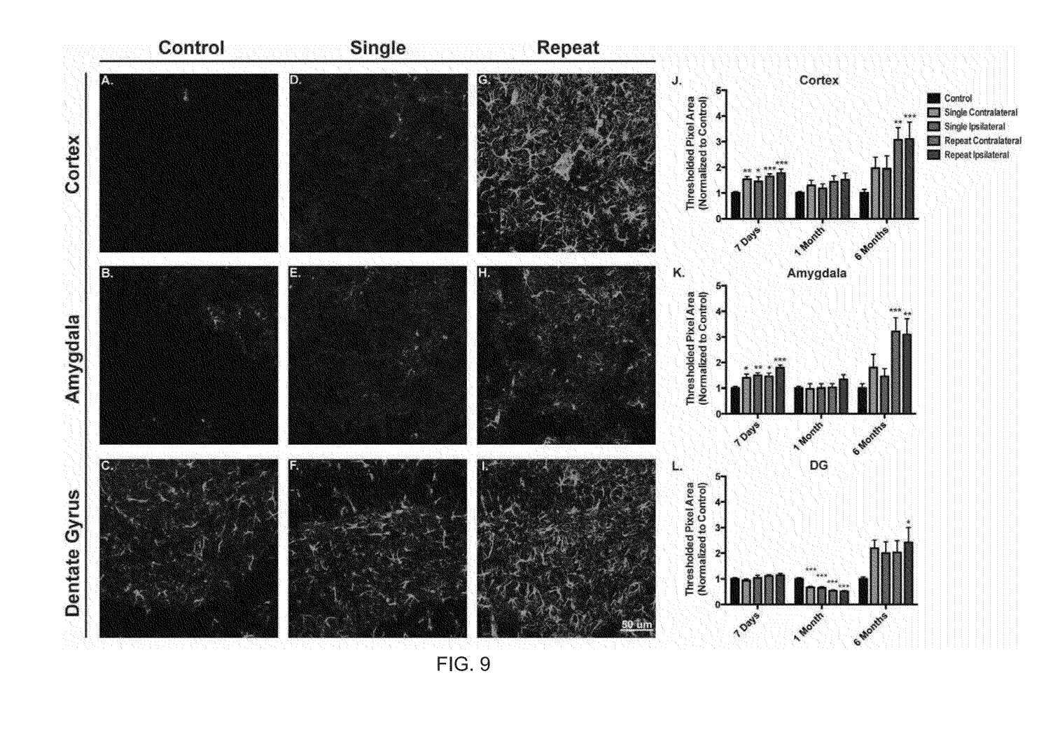

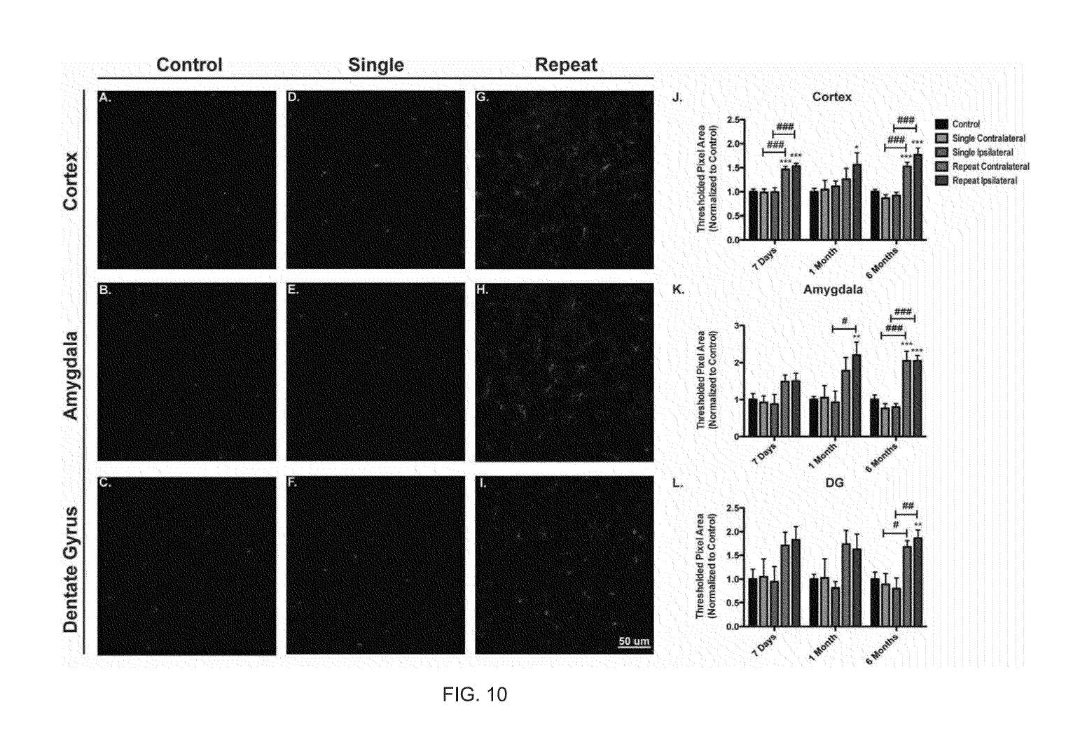

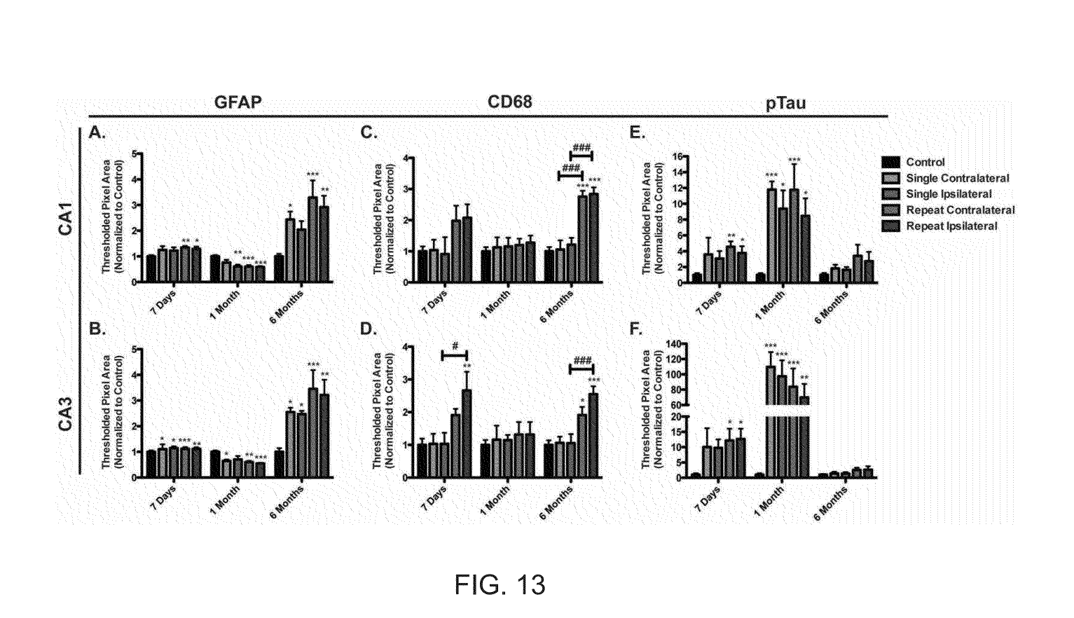

Astrogliosis and increased microglial activation are characteristic of the brains in patients with CTE. We thus sought to characterize the development of reactive astrogliosis (FIG. 9) and microglial activation (FIG. 10) in mice sustaining single and repetitive mTBI in this model at 7 days, 1 month and 6 months post-injury; both ipsilateral and contralateral to the impact site. In the mice receiving a single mTBI, GFAP (reactive astrocytes) labeling was significantly increased compared to control mice at 7 days post-injury in the bilateral cortices (ipsilateral, P=0.03; and contralateral, P=0.005) and amygdalae (ipsilateral, P=0.006; and contralateral, P=0.04) (FIGS. 9J and 9K). By 1 month post-injury the reactive astrogliosis in the cortex and amygdala subsided, with no differences noted between single mTBI and age-matched control mice; however, there was a significant decrease in astrogliosis present in the bilateral DG (ipsilateral and contralateral, P<0.001), ipsilateral CA1 (P=0.002) and contralateral CA3 (P=0.01) regions of the hippocampus (FIGS. 9L and 13). At 6 months, there was only a focal significant elevation in GFAP labeling in the contralateral CA1 (P=0.04) and bilateral CA3 (ipsilateral, P=0.04; and contralateral, P=0.03) regions of the hippocampus (FIG. 13).

In the repetitive mTBI group, there was a significant increase in GFAP labeling at 7 days post-injury in the bilateral cortices (ipsilateral and contralateral, P<0.001) and amygdalae (ipsilateral, P<0.001; and contralateral, P=0.01) (FIGS. 9J and 9K); as well as the bilateral CA1 regions of the hippocampus (ipsilateral, P=0.03; and contralateral, P=0.009) (FIG. 13). Similarly to the single mTBI group, at 1 month there was a resolution of the reactive astrocytosis in the cortex and amygdala; however, interestingly, there was also a significant decrease in astrogliosis present in all regions of both hippocampi: bilateral DG (P<0.001), bilateral CA1 (P<0.001), and bilateral CA3 (ipsilateral, P<0.001; and contralateral, P=0.002) regions (FIG. 13). At 6 months post-injury there was a second and more robust wave of elevated astrogliosis diffusely in the bilateral cortices (ipsilateral, P<0.001; and contralateral, P=0.001) and amygdalae (ipsilateral, P=0.001; and contralateral, P<0.001), as well as increased GFAP labeling in the hippocampi of repetitive mTBI mice compared to age-matched controls: ipsilateral DG (P=0.03), bilateral CA1 (ipsilateral, P=0.003; and contralateral, P<0.001), and bilateral CA3 (ipsilateral, P=0.001; and contralateral, P<0.001) regions (FIGS. 9J-L and 13).

Mice receiving a single mTBI did not exhibit a significant elevation in activated microglia at any of the time-points. On the contrary, the mice in the repetitive mTBI group had microglial activation that increased over time (FIGS. 10J-L and 13). At 7 days post-injury there was a significant limited elevation in microglial activation in the bilateral cortices (P<0.001), as well as the ipsilateral hippocampus (CA1, P=0.005). At 1 month, the microgliosis persisted in the ipsilateral cortex (P=0.04) and amygdala (P=0.005); and by 6 months there was progressive microgliosis involving bilateral cortices (P<0.001), and amygdalae (P<0.001) in repetitive mTBI mice (FIGS. 10J-L). The bilateral hippocampi also exhibited an increase in microglia at 6 months: ipsilateral DG (P=0.009), bilateral CA1 (P<0.001), and bilateral CA3 (ipsilateral, P<0.001; and contralateral, P=0.02) regions (FIGS. 10J-L and 13).

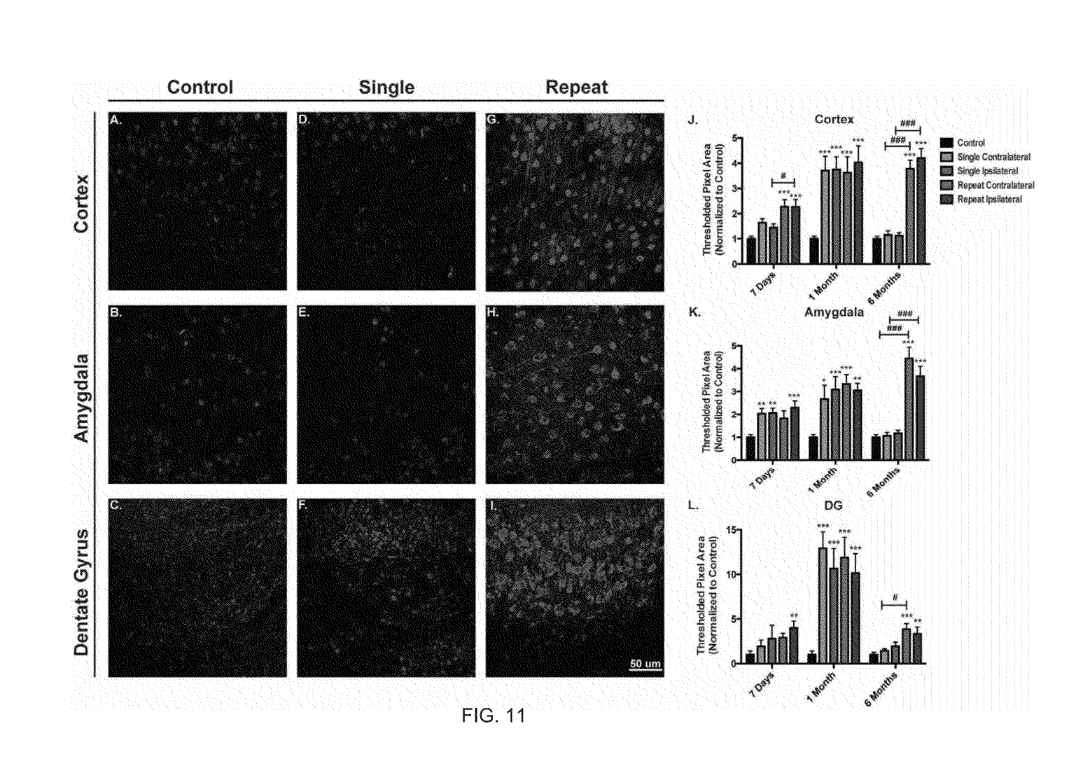

Phosphorylated tau is a histopathological hallmark of CTE and thus we next wanted to explore whether repetitive mTBI would lead to increased tau accumulation in this model. An increase in phosphorylated tau was noted in the single mTBI mice in the amygdala (ipsilateral, P=0.005; and contralateral, P=0.006) at 7 days post-injury and remained elevated and extended to involve the bilateral cortices (P<0.001), amygdalae (ipsilateral, P<0.001; and contralateral, P=0.01), and hippocampi (P<0.001) at 1 month; however, by 6 months no elevation in phosphorylated tau was appreciated (FIGS. 11J-L and 13). On the other hand, in the repetitive mTBI group we observed an increase in phosphorylated tau accumulation at 7 days in the bilateral cortices (P<0.001), the ipsilateral amygdala (P<0.001) and all regions of the hippocampi: ipsilateral DG (P=0.009), bilateral CA1 (ipsilateral, P=0.03; and contralateral, P=0.004) and CA3 (ipsilateral, P=0.01; and contralateral, P=0.02) regions (FIGS. 11J-L and 13). This phosphorylated tau accumulation continued to increase over 1 month post-injury and unlike those mice with a single mTBI, by 6 months we observed a persistent tauopathy in the bilateral cortices (P<0.001), amygdalae (P<0.001) and the DG (ipsilateral, P=0.006; and contralateral, P<0.001) regions of the hippocampi (FIGS. 11J-L).

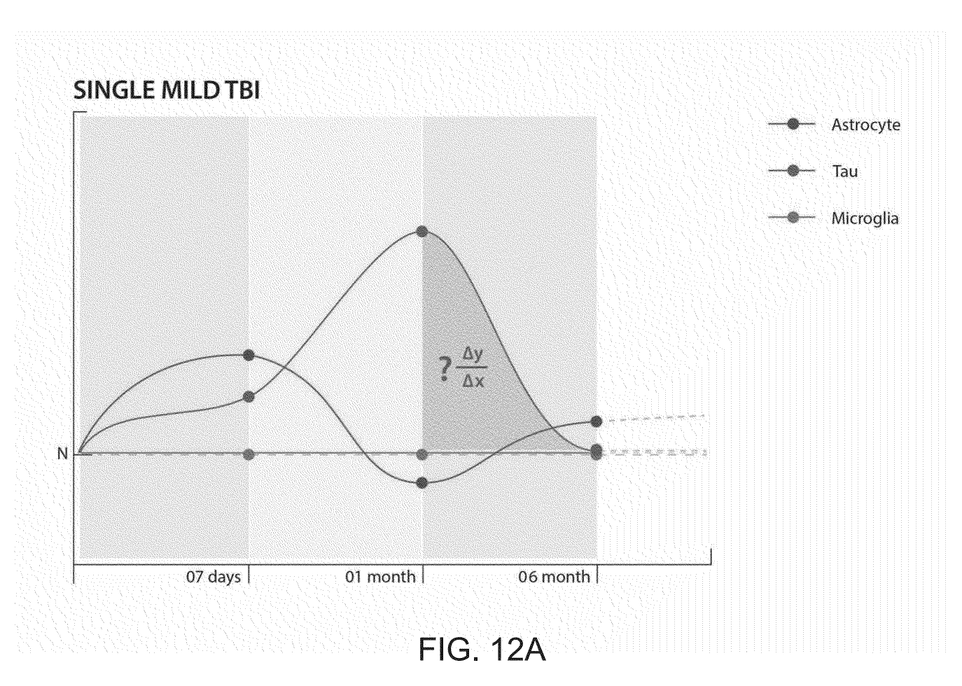

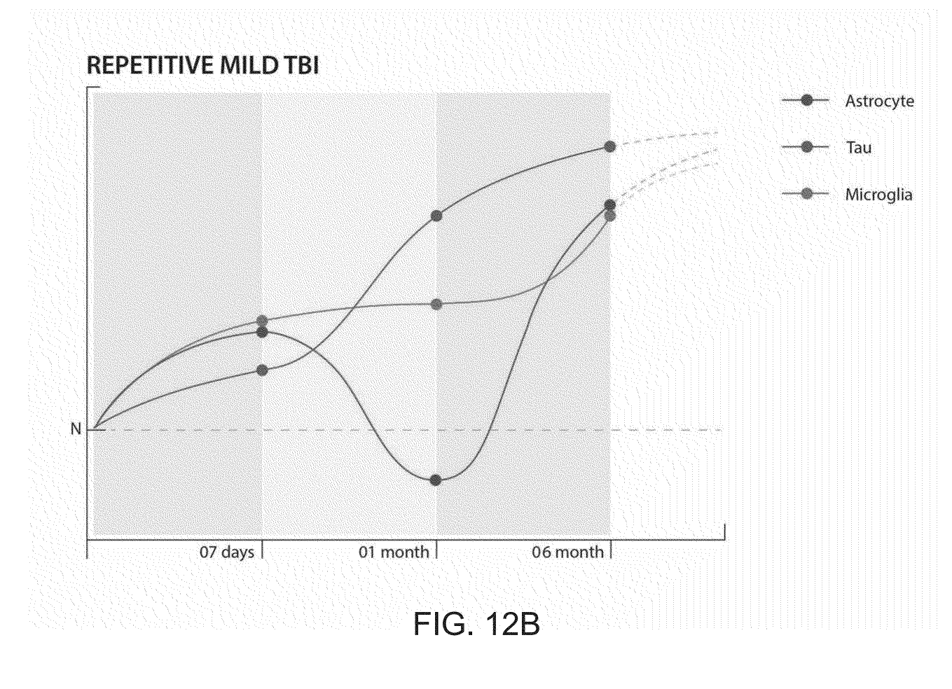

A model of closed head injury can be used to investigate the spectrum of behavioral and neuropathological sequelae following repetitive mild TBI. This model has been developed with several features similar to experience in the clinical setting, including occurring in non-anesthetized animals. With this model, animals exposed to a single mild TBI have short-term “post-concussive” behavioral abnormalities. Based on the histopathological analysis, this is accompanied by a limited acute reactive astrocytosis and phosphorylated tau deposition that is present at 7 days and 1 month but not persistent out to 6 months (FIG. 12). By contrast, the observed cumulative effects of 42 head impacts delivered over the course of seven days recapitulates the neurobehavioral syndrome and characteristic pathological features of CTE. As described in our previous manuscript, these mice exhibit depression, risk-taking behavior, sleep disturbances and persistent cognitive deficits. Pathologically, repetitive mild TBI resulted in a more marked neuro-inflammatory response, with persistent and widespread astrogliosis and microglial activation, as well as a progressive accumulation of phosphorylated tau protein (FIG. 12). Many other studies investigating repetitive mild TBI have demonstrated behavioral deficits and neuropathological changes; however, these changes improve or even resolve with time. Contrary to many of these studies, the changes we observed in the repetitive mild TBI mice progressed and even dynamically changed over a significant length of time.

The spatial distribution of astrogliosis and microglial activation we observed may have numerous causes. While it is possible that the microglial activation and reactive astrocytosis at 6 months is in response to the primary injury, what is more likely is that repetitive mild TBI initiates a progressive, chronic neuro-inflammatory process that contributes to secondary and tertiary injury.

Interestingly, there was a limited acute increase in phosphorylated tau following a single mild TBI that was persistent at 1 month. This parallels findings of hyperphosphorylated tau accumulation acutely following a single experimental rotational or percussion injury; 2 weeks following a single experimental blast injury; and acutely following TBI in humans. The phosphorylated tau accumulation following a single injury seems to clear over time, when animals were analyzed at 6 months. One intriguing observation from our studies in this model was that repetitive mild TBI resulted in a similar elevation of phosphorylated tau; however, this accumulation persisted and increased over time—all in the absence of any further mechanical trauma or outside stress to the animals. This pathological progression has not previously been described in a controlled fashion and lays the foundation for additional analyses elucidating the trigger for delayed tau accumulation and persistent gliosis. It is intriguing to speculate that chronically activated microglia may enhance the hyperphosphorylation of tau and increase the total tau burden, as it has been suggested in other neurodegenerative diseases.

Up to this point much of the focus in CTE has been on the accumulation of phosphorylated tau protein. While the neurotoxicity of phosphorylated tau may contribute to some of the CTE phenotype, in this model, it does not solely account for the behavioral abnormalities observed. If that were the case, the single mTBI mice should have demonstrated more behavioral deficits at 1 month given the elevated phosphorylated tau. Rather, it was only the repetitive mTBI mice in which there was a persistent microglial response. Chronic neuroinflammation may be at the heart of the observed behavioral abnormalities and symptoms following repetitive mTBI. Biochemical, cellular, and animal disease models, as well as clinical studies have elucidated the role of neuroinflammation, particularly microglia, in psychiatric disorders such as depression, PTSD, and bi-polar disorder; which prominently overlap with the behavior observed in CTE and the repetitive mild TBI mice in this model. A recent study demonstrated that the number of TBIs in military personnel was associated with greater suicide risk when controlling for the effects of depression, PTSD, and TBI symptom severity. Those subjects that sustained two or more concussions had a significant increased incidence of suicidal ideation. While difficult to capture suicidal behavior in any animal model, clinical studies have implicated microgliosis and neuroinflammation in the neurobiology of suicide.

With regards to neuro-inflammation, we also observed an interesting finding at 1 month with the GFAP labeling in mild TBI mice. At 1 month, we found a significant decrease in GFAP in all regions of bilateral hippocampi in repetitive TBI mice compared to age-matched control mice. Glial fibrillary acidic protein is a cytoskeletal protein involved in processes related to cell movement and structure and has been proposed to play a role in cell communication such as astrocyte-neuron interactions. Down-regulation of GFAP in the hippocampus has been observed in rodents exposed to chronic social stress. In this pre-clinical model of depression, the authors found that rats in a depressive-like state exhibit reduced GFAP levels in the hippocampal formation. It has been hypothesized that glial dysfunction may play a key role in the pathophysiology of psychiatric disorders, including major depression. Post-mortem clinical studies have shown significantly reduced GFAP immunoreactivity (in cell bodies and fibers) in hippocampal regions CA1 and CA2 of patients with major depression. Our repetitive mild TBI mice demonstrated depressed behavior at the 1-month time-point, and it is intriguing to think that, similar to these other studies, the diffuse reduced hippocampal GFAP we observed may be implicated in this phenotypic behavior. That being said, it is still unknown which presumptive glial changes are causal for depression. Our single mild TBI mice also demonstrated reduced GFAP in some parts of the hippocampus, although they did not exhibit depressed-like behavior on testing at 1-month. It is more likely that glial dysfunction or reduced glial activity is one component contributing to the overall picture and that there are other mechanisms in play that we have yet to fully understand.

The single mild TBI in this model can be classified as a concussion given the few post-concussive behavioral abnormalities observed. Future studies can aim to vary the injury severity; specifically lowering the severity further such that we can investigate the long-term effects of repetitive subconcussion. We chose six hits per day in an attempt to simulate the more frequent head contact seen in contact sports. It may or may not be that less frequent mild TBI would result in the same outcomes. Varying the overall number of head impacts will need to be studied in a dose-dependent fashion; although, the number needed to produce CTE like changes in the mouse will likely not be translatable to humans and overall will be less important than the actual pathophysiological mechanisms leading to post-traumatic neurodegeneration. The model used can facilitate continued investigation contribute to our understanding of the neurological sequelae of repetitive mild TBI, particularly CTE.

As we learn more about the interplay between this dynamic neuroinflammatory response and post-traumatic behavior/neuropathology, new avenues for developing improved diagnostic measures as well as translational treatment approaches for CTE could open up. Whether treatments should be targeting neuroinflammation, tau processing, or mechanisms either upstream or downstream, is currently unknown; and will only become elucidated as we learn more about the pathophysiological effects of repetitive mild TBI. This model will allow for a controlled, mechanistic analysis of CTE in the future, because it is the first to encapsulate the spectrum of this human phenomenon.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

A phrase such as “an aspect” does not imply that such aspect is essential to the subject technology or that such aspect applies to all configurations of the subject technology. A disclosure relating to an aspect may apply to all configurations, or one or more configurations. An aspect may provide one or more examples of the disclosure. A phrase such as “an aspect” may refer to one or more aspects and vice versa. A phrase such as “an embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples of the disclosure. A phrase such “an embodiment” may refer to one or more embodiments and vice versa. A phrase such as “a configuration” does not imply that such configuration is essential to the subject technology or that such configuration applies to all configurations of the subject technology. A disclosure relating to a configuration may apply to all configurations, or one or more configurations. A configuration may provide one or more examples of the disclosure. A phrase such as “a configuration” may refer to one or more configurations and vice versa.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

As used herein, the phrase “at least one of” preceding a series of items, with the terms “and” or “or” to separate any of the items, modifies the list as a whole, rather than each member of the list (i.e., each item). The phrase “at least one of” does not require selection of at least one item; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Furthermore, to the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

While certain aspects and embodiments of the invention have been described, these have been presented by way of example only, and are not intended to limit the scope of the invention. Indeed, the novel methods and systems described herein may be embodied in a variety of other forms without departing from the spirit thereof. The accompanying claims and their equivalents are intended to cover such forms or modifications as would fall within the scope and spirit of the invention.

What is claimed is:

1. A method of modeling traumatic brain injury, comprising:
inserting a subject into a sheath, such that a head of the subject is restrained relative to a body of the subject;
placing the subject, while in the sheath, on a pad beneath an impactor;
impacting the head by the impactor, such that the head moves into the pad in response to the impacting.

2. The method of claim 1, wherein the subject is not anesthetized during the impacting.

3. The method of claim 1, wherein the head is unbound relative to the pad during the impacting.

4. The method of claim 1, wherein the inserting comprises:
placing the subject through a first opening at the first end of the sheath; and
closing the first end to limit retraction of the subject through the first opening.

5. The method of claim 1, further comprising, prior to the impacting, placing a helmet on the head, the helmet comprising an impact plate.

6. The method of claim 5, further comprising, prior to the impacting, aligning the impact plate at an impact location at the head.

7. The method of claim 6, wherein the head is impacted by transferring a force received from the impactor through the impact plate to the head.

8. An impact model system, comprising:
a pad configured to support a subject and allow a range of travel by a head of the subject into the pad after an impact;
an impactor having a tip configured to travel from an initial location to an impact location at the head;
a containment sheath configured to restrain the head relative to a body of the subject.

9. The impact model system of claim 8, wherein the sheath comprises a first opening at a first end of the sheath, configured to receive the subject into the sheath.

10. The impact model system of claim 9, wherein the first opening is configured to be closed to prevent retraction through the first opening.

11. The impact model system of claim 8, wherein the sheath comprises a second opening at a second end of the sheath, configured to provide an airway for the subject.

12. The impact model system of claim 8, further comprising a helmet configured to be placed on the head and received the impact from the impactor.

13. The impact model system of claim 12, wherein the helmet comprises an impact plate configured to be aligned at the impact location.

14. The impact model system of claim 12, wherein the helmet comprises a band configured to secure the helmet to the head.

15. The impact model system of claim 12, wherein the helmet comprises a handle configured to receive a magnetic connection.

16. The impact model system of claim 15, further comprising an arm configured to form a magnetic connection with the handle.

17. The impact model system of claim 16, further comprising an arm configured to release the magnetic connection upon advancement of the impactor from the initial location to the impact location.

18. A method, comprising:
placing a subject on a pad beneath an impactor, the subject having a helmet attached to a head of the subject;
connecting an arm of an impact device to a handle of the helmet, such that the head is restrained relative to the impact device;
impacting the head by advancing the impactor from an initial location to an impact location;
while advancing the impactor, disconnecting the arm from the handle, such that the head becomes unrestrained relative to the impact device and moves into the pad in response to the impacting.

19. The method of claim 18, wherein the subject is not anesthetized during the impacting.

20. The method of claim 18, wherein the connecting comprises applying a magnetic force between the arm and a handle.

21. The method of claim 20, wherein the disconnecting comprises applying a force to the arm that exceeds the magnetic force.
